(19) United States
(12) Patent Application Publication
(10) Pub. No.: US 2025/0195090 A1
(43) Pub. Date: Jun. 19, 2025

(54) CATHETER CLOG DETECTION IN THROMBECTOMY SYSTEMS

(71) Applicant: Walk Vascular, LLC, Irvine, CA (US)

(72) Inventors: Seung Hae Clare CHUNG, Los Altos, CA (US); Jared Ryan G. LUMAUIG, San Jose, CA (US)

(21) Appl. No.: 18/968,851

(22) Filed: Dec. 4, 2024

Related U.S. Application Data
(60) Provisional application No. 63/610,247, filed on Dec. 14, 2023.

(51) Int. Cl. A61B 17/22; A61B 17/00; A61B 90/00

(52) U.S. Cl. CPC A61B 17/22; A61B 17/00234; A61B 90/06; A61B 2017/00039; A61B 2017/00057; A61B 2017/00092; A61B 2017/00106; A61B 2017/00119; A61B 2017/00238; A61B 2017/22079; A61B 2090/064; A61B 2217/005; A61B 2217/007

(57) ABSTRACT

A thrombectomy system includes an aspiration catheter configured for advancement through vasculature of a subject to facilitate clot removal from the vasculature of the subject. The thrombectomy system includes a fluid jet proximate to a distal end of the aspiration catheter. The thrombectomy system includes a flow direction sensor positioned on the aspiration catheter proximate to the fluid jet. The flow direction sensor is configured to detect a flow direction of fluid at a distal region of the aspiration catheter during operation of a clot removal state of the thrombectomy system. The flow direction of the fluid at the distal region of the aspiration catheter indicates a clog state of the aspiration catheter.

### CROSS-REFERENCE TO RELATED APPLICATION(S)

This application claims priority to U.S. Provisional Patent Application No. 63/610,247, filed on Dec. 14, 2023, and entitled “CATHETER CLOG DETECTION IN THROMBECTOMY DEVICES”, the entirety of which is incorporated herein by reference for all purposes.

### BACKGROUND

### Technical Field

The present disclosure pertains generally to medical devices and methods of their use. More particularly, the present invention pertains to aspiration and thrombectomy devices and methods of use thereof.

### Description of the Related Technologies

Blood clots (thrombi) can form in various parts of the body and can pose a serious health risk. For instance, blood clots can block blood flow and/or lead to tissue damage, organ dysfunction, or life-threatening conditions like stroke or heart attack. Thrombectomy is a medical procedure used to remove blood clots from blood vessels to restore blood flow and prevent further complications.

Various types of catheter-based thrombectomy devices have been developed to aid in the removal of thrombotic material. Such devices are typically inserted into the affected blood vessel through a small incision or artery access point. Catheter-based thrombectomy devices include mechanical thrombectomy devices, rheolytic thrombectomy devices, and others (e.g., ultrasound-assisted devices).

Mechanical thrombectomy devices can implement various types of mechanical components to engage with and remove thrombotic material. For instance, stent retrievers and clot retriever baskets are designed for navigation through vasculature to the site of a clot, deployment at the clot site to cause the stent retriever or clot retriever basket to entrap the clot, and withdrawal through the vasculature to facilitate clot removal. As another example, suction-based thrombectomy devices use negative pressure to aspirate clots from blood vessels (e.g., via a catheter with a distal tip to be placed near the clot prior to activation to draw the clot into a collection chamber, where it is trapped and removed). As yet another example, rotational thrombectomy devices employ rotational mechanisms to fragment and remove clots (e.g., a rotating wire or catheter tip for creating shear forces that break down clots), allowing the fragments to be cleared by the body or using aspiration or other techniques.

Rheolytic thrombectomy devices employ mechanisms that rely on high-velocity jets to break down and remove thrombotic material. Rheolytic thrombectomy mechanisms may be positioned on catheters (e.g., at or near the distal tip) and can utilize saline solution, or a mixture of saline and the patient's own blood, to create high-velocity jets for direction toward clots to generate shear forces that disrupt the clot's structure. The jetted fluid can cause fragmentation of the clot, and the fragments may then be cleared naturally from the body or by aspiration techniques.

Some thrombectomy devices employ aspects of suction-based thrombectomy devices and rheolytic thrombectomy devices. For instance, some thrombectomy devices utilize a saline jet positioned at or near a distal tip of an aspiration catheter, allowing for aspiration of clot fragments as the jetted saline macerates the clot (e.g., thrombus and/or soft emboli).

Catheter-based thrombectomy devices sometimes experience catheter clogging during clot removal operations. Catheter clogging can result from attempting to aspirate large or tough clots that can be resistant to fragmentation (e.g., due to calcification or fibrin richness). Catheter clogging during clot removal operations can often go undetected (e.g., unless the clinician pays close attention to canister blood flow). When the catheter is clogged, suction at the distal tip of the catheter can be lost, and fluid can be sprayed out from the distal tip of the catheter (when a saline jet is present). Fluid released from the distal tip of the catheter can cause cell lysis and/or displacement of blood cells. Additionally, fluid released from the distal tip of the catheter can cause clot material to be pushed away from or spin about the distal tip of the catheter, which can further complicate and/or prolong a clot removal procedure.

The subject matter disclosed herein is not limited to embodiments that operate only in environments such as those described above. Rather, this background is only provided to illustrate one example technology area where some embodiments described herein may be practiced.

### SUMMARY

At least some disclosed embodiments provide a thrombectomy system that includes (i) an aspiration catheter configured for advancement through vasculature of a subject to facilitate clot removal from the vasculature of the subject; (ii) a fluid jet proximate to a distal end of the aspiration catheter; and (iii) a flow direction sensor positioned on the aspiration catheter proximate to the fluid jet. The flow direction sensor is configured to detect a flow direction of fluid at a distal region of the aspiration catheter during operation of a clot removal state of the thrombectomy system. The flow direction of the fluid at the distal region of the aspiration catheter

At least some disclosed embodiments provide a thrombectomy system that includes (i) an aspiration catheter configured for advancement through vasculature of a subject to facilitate clot removal from the vasculature of the subject; (ii) a fluid jet proximate to a distal end of the aspiration catheter; and (iii) a flow sensor positioned on the aspiration catheter proximal to the fluid jet. The flow sensor is configured to detect a flow rate of fluid within an aspiration lumen of the aspiration catheter during operation of a clot removal state of the thrombectomy system. The flow rate of the fluid within the aspiration lumen indicates a clog state of the aspiration catheter.

This summary is provided to introduce a selection of concepts in a simplified form that are further described below in the detailed description. This summary is not intended to identify key features or essential features of the claimed subject matter, nor is it intended to be used as an indication of the scope of the claimed subject matter.

### BRIEF DESCRIPTION OF THE DRAWINGS

Various objects, features, characteristics, and advantages of the invention will become apparent and more readily appreciated from the following description of the embodiments, taken in conjunction with the accompanying drawings and the appended claims, all of which form a part of this specification. In the Drawings, like reference numerals may be utilized to designate corresponding or similar parts in the various Figures, and the various elements depicted are not necessarily drawn to scale, wherein:

FIG. 1 illustrates a perspective view of an example aspiration catheter.

FIG. 2 illustrates a plan view of example disposable components of a system for aspirating thrombus according to an embodiment of the present disclosure.

FIG. 3 illustrates a sectional view of an example distal end of the aspiration catheter of the system for aspirating thrombus of FIG. 1.

FIG. 4 illustrates a detail view of an example y-connector of the aspiration catheter of the system for aspirating thrombus of FIG. 1.

FIG. 5 illustrates a plan view of example disposable components of a system for aspirating thrombus according to an embodiment of the present disclosure.

FIG. 6 illustrates a perspective view of an example system for aspirating thrombus of FIG. 4

FIGS. 7A, 7B, 8A, 8B, 9A, 9B, 10A, 10B, 11A, and 11B illustrate conceptual representations of a thrombectomy system that includes a flow direction sensor positioned near a fluid jet of an aspiration catheter, according to implementations of the present disclosure.

FIGS. 12A, 12B, 13A, 13B, 14A, and 14B illustrate conceptual representations of a thrombectomy system that includes a flow sensor positioned proximal to a fluid jet of an aspiration catheter, according to implementations of the present disclosure.

### DETAILED DESCRIPTION

As indicated hereinabove, catheter clogging incidents in catheter-based thrombectomy systems can often go undetected. For example, many aspiration catheters lack a mechanism to determine whether a catheter of a thrombectomy system is clogged during clot removal operations. To detect catheter clogging during clot removal operations, users often rely on careful observation of canister blood flow, which can detract from the user's attention to other aspects of a clot removal operation. Failure to detect a clogged catheter during a clot removal operation can result in blood lysis, displacement of blood cells, clot displacement, and/or other negative outcomes for patients.

The present disclosure pertains to systems, devices, and techniques for detecting catheter clogging in thrombectomy systems.

For example, a thrombectomy system may include a flow direction sensor position near the fluid jet at the distal end of the aspiration catheter. The flow direction sensor may take on various forms, such as an ultrasonic flow meter, an electromagnetic flow meter, a temperature-based flow meter, an optical-based flow meter (e.g., to determine hemoglobin levels), and/or others. The flow direction sensor can be configured to detect the flow direction of fluid at the distal region of the aspiration catheter. Detection of flow in a proximal direction (e.g., into and/or through the aspiration catheter toward the canister) can indicate the absence of catheter clogging. In contrast, detection of flow in a distal direction (e.g., outward from the aspiration catheter and away from the canister) can indicate the presence of catheter clogging. Based on a catheter clogging state indicated via the flow direction sensor, the thrombectomy system can selectively deactivate clot removal operations and/or present an alert to a user (e.g., directing the user to disable the clot removal state, or advising the user that a clog may be present).

An additional or alternative flow meter may be positioned within the aspiration lumen of the aspiration catheter of the thrombectomy system. The flow meter may detect the flow rate of fluid within the aspiration lumen during clot removal operations. The flow rate within the aspiration lumen can indicate the clog state of the aspiration catheter. For instance, detection of a flow rate below certain levels (e.g., after a startup routine or time period, or after initially reaching a minimum flow rate) can indicate the presence of catheter clogging, whereas detection of a flow rate above certain levels (e.g., after the startup routine, time period, or reaching the minimum flow rate) can indicate the absence of catheter clogging. Based on the catheter clogging state indicated via the flow meter, the thrombectomy system can selectively deactivate clot removal operations and/or present an alert to a user (e.g., directing the user to disable the clot removal state, or advising the user that a clog may be present).

In some instances, implementing a clog detection system on a thrombectomy device, as described herein, may provide various advantages, such as reducing the duration of thrombectomy operations, reducing clot movement/displacement during thrombectomy operations, and others. Such advantages can beneficially facilitate improved patient outcomes.

Although examples discussed herein focus, in at least some respects, on implementing a clog detection system on an aspiration catheter that includes a fluid jet at the distal region, the techniques and/or components discussed herein may be implemented on aspiration catheters that omit fluid jets, and/or on other types of catheter-based devices, even outside of the domain of thrombectomy.

### Example Thrombectomy Device

The following discussion relates to an example catheter-based thrombectomy device that implements aspiration aspects and rheolytic aspects and that may comprise or be used to implement at least some disclosed embodiments. As noted above, the principles disclosed herein may be implemented in conjunction with other types of catheter-based thrombectomy systems.

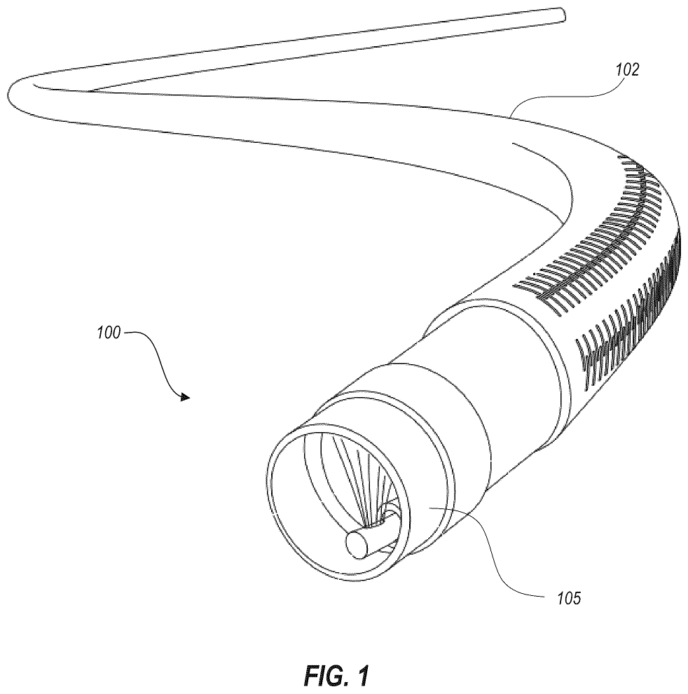

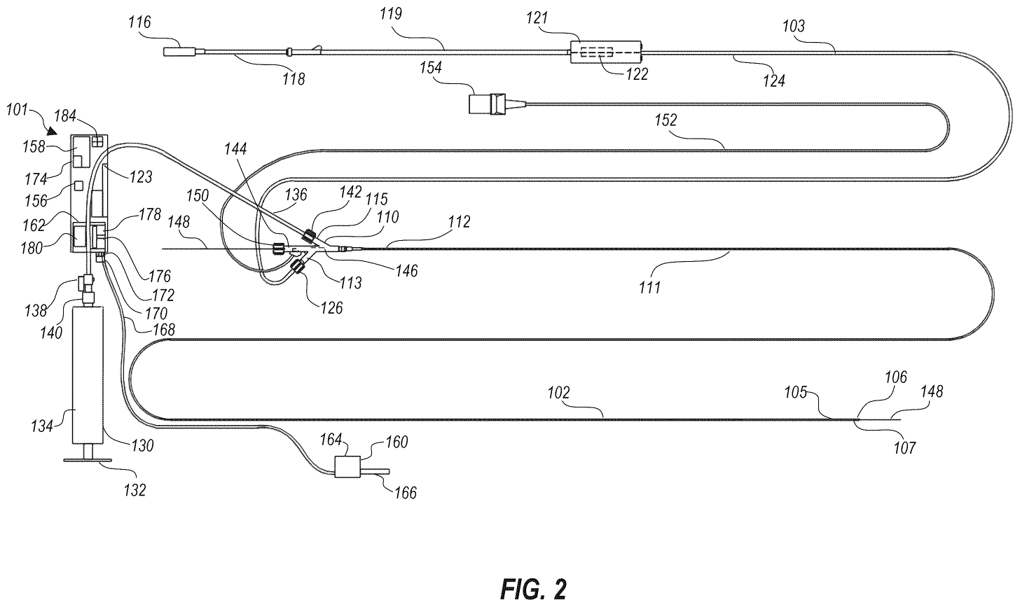

A system 100 for aspirating thrombus is illustrated in FIG. 1, illustrating primarily a distal end 105 of an aspiration catheter 102. FIGS. 2-4 illustrate the system 100 in greater detail. The system 100 for aspirating thrombus includes three major components: a pump 101, an aspiration catheter 102, and a tubing set 103. The aspiration catheter 102 and the tubing set 103 may comprise disposable components. The pump 101 and the pump's associated pump base may comprise reusable components. In some implementations, it is not necessary to sterilize the pump 101, as it may be kept in a non-sterile field or area during use. The aspiration catheter 102 and the tubing set 103 may each be supplied sterile, after sterilization by ethylene oxide gas, electron beam, gamma, or other sterilization methods. The aspiration catheter 102 may be packaged and supplied separately from the tubing set 103, or the aspiration catheter 102 and the tubing set 103 may be packaged together and supplied together. Alternatively, the aspiration catheter 102 and tubing set 103 may be packaged separately, but supplied together (i.e., bundled).

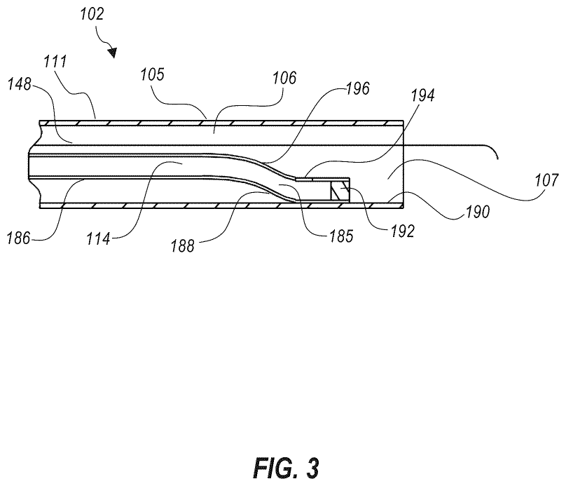

As shown in FIGS. 2-4, the aspiration catheter 102 has a distal end 105 and includes an over-the-wire guidewire lumen/aspiration lumen 106 extending between an open distal end 107, and a proximal end comprising a y-connector 110. The catheter shaft 111 of the aspiration catheter 102 is connected to the y-connector 110 via a protective strain relief 112. In other embodiments, the catheter shaft 111 may be attached to the y-connector 110 with a luer fitting. The y-connector 110 comprises a first female luer 113 which communicates with a catheter supply lumen 114 (FIG. 3), and a second female luer 115 which communicates with the guidewire lumen/aspiration lumen 106.

A spike 116 for coupling to a fluid source (e.g., saline bag, saline bottle) allows fluid to enter through an extension tubing 118 and flow into a supply tube 119. An optional injection port allows injection of materials or removal of air. A cassette 121 having a moveable piston 122 is used in conjunction with a mechanical actuator 123 of the pump 101. Fluid is pumped into an injection tube 124 from action of the cassette 121 as applied by the actuator 123 of the pump 101. A male luer 126, hydraulically communicating with the catheter supply lumen 114, via the injection tube 124, is configured to attach to the female luer 113 of the y-connector 110.

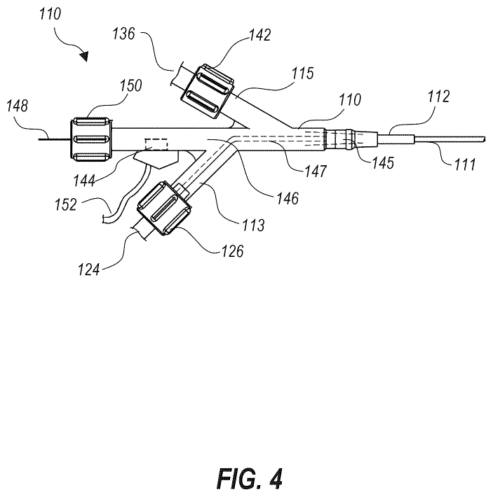

Accessories are illustrated that are intended for applying a vacuum source, such as a syringe 130 having a plunger 132 and a barrel 134, to the aspiration lumen 106 of the catheter 102. The syringe 130 is attached to a vacuum line 136 via the luer 140 of the syringe 130. A stopcock 138 may be used on the luer 140 to maintain the vacuum, or alternatively, the plunger 132 may be a locking variety of plunger that is configured to be locked in the retracted (vacuum) position. A male luer 142 at the end of the vacuum line 136 may be detachably secured to the female luer 115 of the y-connector 110 of the aspiration catheter 102. As shown in more detail in FIG. 4, a pressure sensor or transducer 144 is secured inside an internal cavity 146 of the y-connector 110 proximal to the female luer 113 and the female luer 115. A valve 150, for example a Touhy-Borst, at the proximal end of the y-connector 110 allows hemostasis of the guidewire lumen/aspiration lumen 106 around a guidewire 148. In other embodiments, the valve 150 may comprise a longitudinally spring-loaded seal. The guidewire 148 may be inserted entirely through the guidewire lumen/aspiration lumen 106. Signals output from the pressure sensor 144 are carried through a cable 152 to a connector 154. The connector 154 is plugged into a socket 156 of the pump 101. Pressure related signals may be processed by a circuit board 158 of the pump 101. The pressure transducer 144 may be powered from the pump 101, via the cable 152. The accessories may also be supplied sterile to the user.

A foot pedal 160 is configured to operate a pinch valve 162 for occluding or opening the vacuum line 136. The foot pedal 160 comprises a base 164 and a pedal 166 and is configured to be placed in a non-sterile area, such as on the floor, under the procedure table/bed. The user steps on the pedal 166 causing a signal to be sent along a cable 168 which is connected via a plug 170 to an input jack 172 in the pump 101. The vacuum line 136 extends through a portion of the pump 101. The circuit board 158 of the pump may include a controller 174 configured to receive one or more signals indicating on or off from the foot pedal 160. The controller 174 of the circuit board 158 may be configured to cause an actuator 176 carried by the pump 101 to move longitudinally to compress and occlude the vacuum line 136 between an actuator head 178 attached to the actuator 176 and an anvil 180, also carried by the pump 101. By stepping on the pedal 166, the user is able to thus occlude the vacuum line 136, stopping the application of a negative pressure. In some embodiments, as the pedal 166 of the foot pedal 160 is depressed, the controller may be configured to open the pinch valve 162.

The pressure transducer 144 thus senses a negative pressure and sends a signal, causing the controller to start the motor of the pump 101. As the effect via the electronics is substantially immediate, the motor starts pumping almost immediately after the pedal 166 is depressed. As the pedal 166 of the foot pedal 160 is released, the controller 174 then causes the pinch valve 162 to close. The pressure transducer 144 thus senses that no negative pressure is present and the controller 174 causes the motor of the pump 101 to shut off. Again, the effect via the electronics is substantially immediate, and thus the motor stops pumping almost immediately after the pedal 166 is depressed. During sterile procedures, the main interventionalist is usually “scrubbed” such that the hands only touch items in the sterile field. However, the feet/shoes/shoe covers are not in the sterile field. Thus again, a single user may operate a switch (via the pedal 166) while also manipulating the catheter 102 and guidewire 148. However, this time, it is the sterile field hands and non-sterile field feet that are used. Alternatively, the foot pedal 160 may comprise two pedals, one for occlude and one for open. In an alternative foot pedal embodiment, the pedal 166 may operate a pneumatic line to cause a pressure activated valve or a cuff to occlude and open the vacuum line 136, for example, by forcing the actuator head 178 to move. In another alternative embodiment, the pedal 166 may turn, slide, or otherwise move a mechanical element, such as a flexible pull cable or push rod that is coupled to the actuator 176, to move the actuator head 178. The cable 168 may be supplied sterile and connected to the base 164 prior to a procedure. The occlusion and opening of the vacuum line 136 thus acts as an on and off switch for the pump 101 (via the pressure sensor 144). The on/off function may thus be performed by a user whose hands can focus on manipulating sterile catheters, guidewires, and accessories, and whose foot can turn the pump on and off in a non-sterile environment. This allows a single user to control the entire operation or the majority of operation of the system 100 for aspirating thrombus. This can be an advantage in terms of a rapid, synchronized procedure, but is also helpful in laboratories where additional assistants are not available. The actuator 176 and anvil 180 may be controlled to compress the vacuum line 136 with a particular force, and the actuator 176 may be controlled to move at a particular speed, either when compressing or when removing compression. Speed and force control allows appropriate response time but may also be able to add durability to the vacuum line 136, for example, by not over-compressing. The foot pedal 160 may communicate with the pinch valve 162 via a wired connection through the pump 101 or may communicate with the pinch valve 162 wirelessly. Additionally, or alternatively, the pump may be controlled by buttons 184 or other user interfaces.

It should be noted that in certain embodiments, the pinch valve 162 and the foot pedal 160 may be incorporated for on/off operation of the pinch valve 162 on the vacuum line 136, without utilizing the pressure sensor 144. In fact, in some embodiments, the pressure sensor 144 may even be absent from the system 100 for aspirating thrombus, the foot pedal 160 being used as a predominant control means.

Turning to FIG. 3, a supply tube 186, which contains the catheter supply lumen 114, freely and coaxially extends within the over-the-wire guidewire lumen/aspiration lumen 106. At least a distal end 188 of the supply tube 186 is secured to an interior wall 190 of the guidewire lumen/aspiration lumen 106 of the catheter shaft 111 by adhesive, epoxy, hot melt, thermal bonding, or other securement modalities. A plug 192 is secured within the catheter supply lumen 114 at the distal end 188 of the supply tube 186. The plug 192 blocks the exit of pressurized fluid, and thus the pressurized fluid is forced to exit through an orifice 194 in the wall 196 of the supply tube 186. The free, coaxial relationship between the supply tube 186 and the catheter shaft 111 along their respective lengths, allows for improved flexibility. In some embodiments, in which a stiffer proximal end of the aspiration catheter 102 is desired (e.g., for pushability or even torquability), the supply tube 186 may be secured to the interior wall 190 of the guidewire lumen/aspiration lumen 106 of the catheter shaft 111 along a proximal portion of the aspiration catheter 102, but not along a distal portion. This may be appropriate if, for example, the proximal portion of the aspiration catheter 102 is not required to track through tortuous vasculature, but the distal portion is required to track through tortuous vasculature. The free, substantially unconnected, coaxial relationship between the supply tube 186 and the catheter shaft 111 along their respective lengths, may also be utilized to optimize flow through the guidewire lumen/aspiration lumen 106, as the supply tube 186 is capable of moving out of the way due to the forces of flow (e.g., of thrombus/saline) over its external surface, such that the remaining inner luminal space of the guidewire lumen/aspiration lumen 106 self-optimizes, moving toward the lowest energy condition (least fluid resistance) or toward the largest cross-sectional space condition (e.g., for accommodating and passing pieces of thrombus).

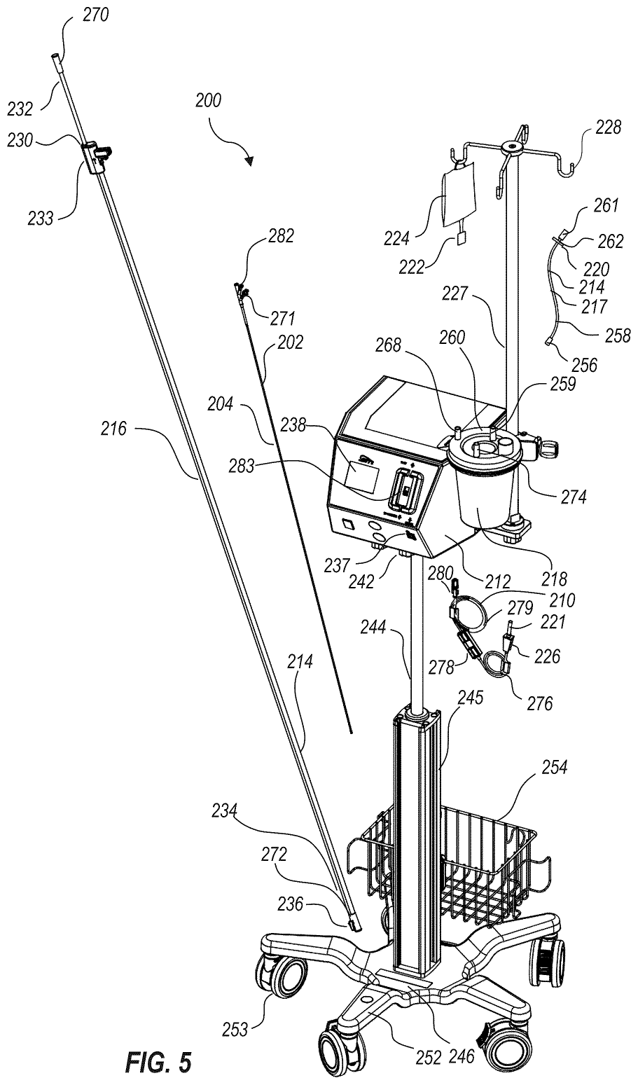

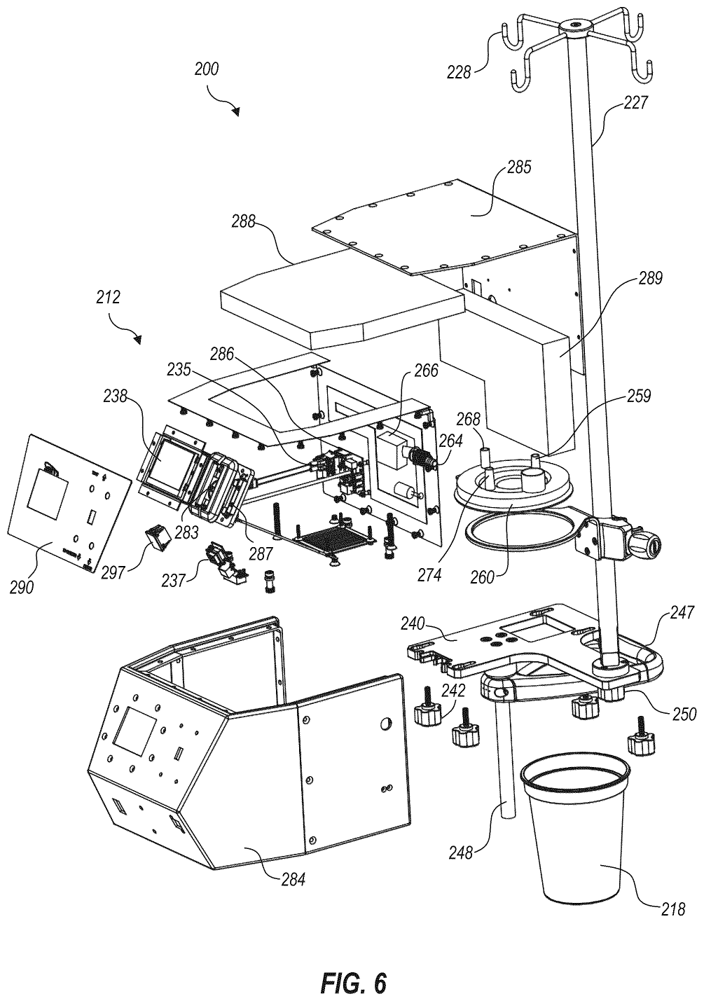

A system 200 for aspirating thrombus is illustrated in FIGS. 5-6. An aspiration catheter 202 is similar to the aspiration catheter 102 of FIGS. 1-4. The aspiration catheter 202 is configured for aspirating thrombus from peripheral vessels, but may also be configured with a size for treating coronary, cerebral, pulmonary or other arteries, or veins. The aspiration catheter 202/system 200 may be used in interventional procedures, but may also be used in surgical procedures. The aspiration catheter 202/system 200 may be used in vascular procedures, or non-vascular procedures (other body lumens, ducts, or cavities). The catheter 202 comprises an elongate shaft 204 configured for placement within a blood vessel of a subject. The catheter 202 may also comprise a catheter supply lumen 114 (FIG. 3) and a guidewire/aspiration lumen 106, each extending along the shaft. The supply lumen 114 may have a proximal end 147 and a distal end 185, and the aspiration lumen 106 may have a proximal end 145 (FIG. 4) and an open distal end 107 (FIG. 3). An orifice or opening 194 may exist at or near the distal end 185 of the supply lumen 114. The orifice or opening 194 may be configured to allow the injection of pressurized fluid into the aspiration lumen 106 at or near the distal end 107 of the aspiration lumen 106 when the pressurized fluid is pumped through the supply lumen 114. In some embodiments, the orifice or opening 194 may be located proximal to the distal end 185 of the supply lumen 114. In some embodiments, the distal end 185 of the supply lumen 114 may comprise a plug 192.

Although examples provided herein focus, in at least some respects, on an aspiration catheter 102 that includes a single orifice 194, an aspiration catheter of a system (e.g., 100 or 200) can include any quantity of orifices through which fluid may pass to form any quantity of fluid jets. For example, a system 100 or 200 for aspirating thrombus can include a quantity of orifices (for forming fluid jets) within a range of 1 to 10, or within a range of 2 to 8, or within a range of 3 to 6.

Although FIG. 3 illustrates the orifice 194 formed in the wall 196 of the supply tube 186, an orifice for forming a fluid jet for an aspiration catheter of a system (e.g., 100 or 200) can be positioned on one or more additional components that are in fluid communication with the supply tube 186. By way of illustrative example, one or more orifices may be formed on a ring (or annulus) that is connected to the catheter shaft 111 (e.g., coaxially connected to the catheter shaft 111 at or near the open distal end 107). The ring can include an internal cavity in fluid communication with the supply tube 186, allowing fluid to travel through the supply tube 186 and the internal cavity of the ring for jetting through the orifice(s) of the ring.

Furthermore, FIG. 3 illustrates the orifice 194 formed as a radial opening in the wall 196 of the supply tube 186, with the orifice wall(s) that forms the orifice 194 between inner and outer surfaces of the supply tube 186 being perpendicular to the tangential axis of the wall 196. In some embodiments, an orifice for forming a fluid jet for an aspiration catheter of a system (e.g., 100 or 200) may be formed as an angled opening, where the orifice wall(s) that forms the orifice (between inner and outer surfaces of a material) has an acute or obtuse angle relative to the tangential axis of the material on which the orifice is formed (e.g., material of a supply tube or an additional component connected to the supply tube). For example, the angle between the orifice wall(s) and the tangential axis of the material on which the orifice is formed can be within a range of 20° to 70°, or 30° to 60°, or 40° to 50° (or within their complementary ranges).

The orifice(s) of an aspiration catheter 102 of a system (e.g., 100 or 200) for providing fluid jets (i.e., “jet-forming” orifices) may be implemented with various sizes or shapes in different embodiments. In one example, an orifice may comprise a circular hole with a diameter between 0.03 mm (0.001 inches) and 0.15 mm (0.006 inches), or between about 0.0508 mm (0.002 inches) and about 0.1016 mm (0.004 inches), or about 0.0787 mm (0.0031 inches). The diameter of the supply lumen 114 may be between about 0.3048 mm (0.012 inches) and about 0.4826 mm (0.019 inches), or between about 0.3556 mm (0.014 inches and about 0.4318 mm (0.017 inches), or about 0.3937 mm (0.0155 inches). As another example, an orifice may comprise a rectangular hole with each side thereof having a length between 0.02 mm (0.0008 inches) and 0.20 mm (0.008 inches). In some implementations, the total cross-sectional area of all jet-forming orifices of an aspiration catheter of a system is between 0.002 mm\hat{ }2 and 0.02 mm\hat{ }2, or between 0.003 mm\hat{ }2 and 0.015 mm\hat{ }2, or between 0.005 mm\hat{ }2 and 0.01 mm\hat{ }2. In some embodiments, pressure measured at the deliver location of each jet-forming orifice during operation of the system (e.g., 100 or 200) is between 400 psi (2.6 MPa) and 2,000 psi (13.8 MPa), or between 500 psi (3.4 MPa) and 2,000 psi (13.8 MPa), or between 600 psi (4.1 MPa) and 1,750 psi (12.1 MPa).

In some implementations, the diameter of the aspiration lumen 106 is within a range of 2 mm (0.08 inches) to 4 mm (0.16 inches). The orifice 194 may be set proximally of the open distal end 107 by a set amount. For example, orifice 194 can be set proximally of the open distal end 107 by about 0.040″ (inches), and in one configuration by 0.051″+−0.003″ or by another desired amount. For example, orifice 42 can be set proximally of the open distal end 107 by approximately 0.035″, 0.036″, 0.037″, 0.038″, 0.039″, 0.040″, 0.041″, 0.042″, 0.043″, 0.044″, 0.045″, 0.046″, 0.047″, 0.048″, 0.049″, 0.050″, 0.051″, 0.052″, 0.053″, 0.054″, 0.055″, 0.056″, 0.057″, 0.058″, 0.059″, 0.060″, or a range defined by any two of the foregoing. In still other configurations, the open distal end 107 can be set proximally of the open distal end 107 by about 0.01″ to about 50″.

In still another configuration a diameter of the aspiration lumen 106 is about 0.075″ (inches) to about 0.177″ (inches), the orifice 194 (and so a distal end of the supply tube 186) can be set proximally of open distal end 107 about 12″ (inches). In still another configuration a diameter of the aspiration lumen 106 is about 0.075″ (inches), the orifice 194 (and so a distal end of the supply tube 186) can be set proximally of open distal end 107 about 0.035″ (inches) to about 0.060″ (inches). In still another configuration, the orifice 194 (and so a distal end of the supply tube 186) can be set proximally of open distal end 107 about 0.010″ (inches) to about 50″ (inches) where the catheter 102 has a catheter size ranging from about 3 Fr to about 50 Fr. In still another configuration, the orifice 194 (and so a distal end of the supply tube 186) can be set proximally of open distal end 107 about 0.010″ (inches) to about 0.200″ (inches), from about 0.010″ (inches) to about 2″ (inches), 0.010″ (inches) to about 40″ (inches), 0.010″ (inches) to about 50″ (inches), or a range defined by any two of the foregoing. In still other configurations, the orifice 194 (and so a distal end of the supply tube 186) can be set proximally of open distal end 107 based upon Table 1:

| | Distal Orifice Proximal |
|---|---|
| Catheter size | Offset (range in inches) |
| 3 Fr | .01″ to .600″ |
| 4 Fr | |
| 5 Fr | |
| 6 Fr | |
| 7 Fr | |
| 8 Fr | |
| 9 Fr | 0.01″ to 40″ |
| 10 Fr | |
| 11 Fr | |
| 12 Fr | .01″ to 40″ |
| 13 Fr | |
| 14 Fr | |
| 15 Fr | |
| 16 Fr | |
| 17 Fr | .01″ to 50″ |
| 18 Fr | |
| 19 Fr | |
| 20 Fr | |
| 21 Fr | |
| 22 Fr | |
| 23 Fr | |
| 24 Fr | |
| 25 Fr | |
| 26 Fr | |

In another configuration, the orifice 194 (and so a distal end of the supply tube 186) can be set proximally of open distal end 107 about 0.010″ (inches) to about 0.200″ (inches) for a catheter having a catheter size ranging from about 3 Fr to about 8 F. In another configuration, the orifice 194 (and so a distal end of the supply tube 186) can be set proximally of open distal end 107 about 0.010″ (inches) to about 2″ (inches) for a catheter having a catheter size ranging from about 9 Fr to about 11 Fr. In another configuration, the orifice 194 (and so a distal end of the supply tube 186) can be set proximally of open distal end 107 about 0.010″ (inches) to about 40″ (inches) for a catheter having a catheter size ranging from about 12 Fr to about 16 F. In another configuration, the orifice 194 (and so a distal end of the supply tube 186) can be set proximally of open distal end 107 about 0.010″ (inches) to about 50″ (inches) for a catheter having a catheter size ranging from about 17 Fr to about 26 F. In other configurations, the ranges can include any combination of a range defined by any two of the foregoing proximal location of the orifice 194 with a range defined by any two of the foregoing catheter sizes.

A pump set 210 (e.g., tubing set) is configured to hydraulically couple the supply lumen 114 to a pump within a saline drive unit (SDU) 212, for injecting pressurized fluid (e.g., saline, heparinized saline) through the supply lumen 114. Suction tubing 214, comprising sterile suction tubing 216 and non-sterile suction tubing 217, is configured to hydraulically couple a vacuum canister 218 to the aspiration lumen 106. A filter 220 may be carried in-line on the suction tubing 214, for example, connected between the sterile suction tubing 216 and the non-sterile suction tubing 217, or on the non-sterile suction tubing 217. The filter 220 is configured to capture large elements such as large pieces of thrombus or emboli.

The pump set 210 includes a saline spike 221 for connection to a port 222 of a saline bag 224, and an inline drip chamber 226 for visually assessing the movement of saline, as well as keeping air out of the fluid being injected. The saline bag 224 may be hung on an IV pole 227 on one or more hooks 228. A pressure sensor 230 such as a vacuum sensor may be used within any lumen of the pump set 210, the suction tubing 214, the supply lumen 114 or aspiration lumen 106 of the catheter 202, or any other component which may see fluid flow. Additional or alternative pressure sensors may be implemented to measure pressure associated with the vacuum canister 218. The pressure sensor 230 is shown in FIG. 5 within a lumen at a junction between a first aspiration tube 232 and a control 233. A cable 234 carries signals output from the pressure sensor 230 to a controller 235 in the SDU 212. A connector 236, electrically connected to the cable 234, is configured to be detachably coupled to a mating receptacle 237 (e.g., input jack) in the SDU 212. The SDU 212 also may have a display 238, including an LCD screen or alternative screen or monitor, in order to visually monitor parameters and status of a procedure. In some instances, one or more fluid flow sensors is/are utilized in addition to or as an alternative to the pressure sensor 230. In some embodiments, the fluid flow sensor is a Doppler flow velocity sensor, or other type of flow sensor. In some instances, flow metrics may be inferred or characterized by implementing multiple pressure sensors (e.g., (i) a pressure sensor on the pump set 210, suction tubing 214, or aspiration lumen 106, and (ii) a pressure sensor on the vacuum canister 218).

In the example of FIGS. 5 and 6, the SDU 212 is held on a mount 240 by four locking knobs 242. The mount 240 is secured to a telescoping rod 244 that is adjustable from a cart base 245 via a cart height adjustment knob or other element 246. The mount 240 and a handle 247 are secured to the rod 244 via an inner post 248 that is insertable and securable within an inner cavity in the rod 244. The IV pole 227 secures to the mount 240 via a connector 250. The base 245 may include legs 252 having wheels 253 (e.g., three or more wheels or four or more wheels) and may be movable via the handle 247. The system 200 may also carry a basket 254 for placement of components, products, documentation, or other items.

In use, a user connects a first connector 256 at a first end 258 of the non-sterile suction tubing 217 to a port 259 on the lid 260 of the canister 218, and connects a second connector 261 at a second end 262 of the non-sterile suction tubing 217 to a vacuum pump input 264 in the SDU 212. A vacuum pump 266 may be carried within the SDU 212 in order to maintain a vacuum/negative pressure within the canister 218. Alternatively, the vacuum inside the canister 218 may be maintained manually, without a vacuum pump, by evacuating the canister 218 via one or more additional ports 268. A user connects a first connector 270 of the sterile suction tubing 216 to an aspiration luer 271 of the aspiration catheter 202 (similar to luer 115), and connects the second connector 272 of the sterile suction tubing 216 to port 274 in the lid 260 of the canister 218. Connector 236 is then coupled to the mating receptacle 237 in the SDU 212 for communication with the control 233 and/or the pressure sensor 230. For instance, the connector 236 can be snapped into mating receptacle 237 in the SDU 212 for communication with elements of the control 233 and/or for communication with the pressure sensor 230, either via cable 234, and/or additional cables or wires. Alternatively, the connector 236 may couple to the mating receptacle 237 by clipping, friction fitting, vacuum fitting, or other means.

After allowing saline to purge through the supply tube 276, cassette 278, and injection tube 279 of the pump set 210, the user connects the luer connector 280 of the pump set 210 to a luer 282 of the aspiration catheter 202 (similar to luer 113). The cassette 278 (similar to cassette 121) is then attached to a saddle 283 in the SDU 212. The saddle 283 is configured to reciprocate a piston to inject the saline from the IV bag 224 at high pressure, after the cassette 278 is snapped in place, keeping the internal contents (e.g., saline) sterile. Systems configured for performing this type of sterile injection of high-pressure saline are described in U.S. Pat. No. 9,883,877, issued Feb. 6, 2018, and entitled, “Systems and Methods for Removal of Blood and Thrombotic Material”, which is incorporated by reference in its entirety for all purposes. The SDU 212 is enclosed within a case 284 and a case lid 285. The controller 235 may reside on a circuit board 286. Noise from a motor 287 controlling the saddle 283 and from the vacuum pump 266 may be abated by internal foam sections 288, 289. The saddle 283 may be moved directly by the motor 287, or may be moved with pneumatics, using a cycled pressurization. An interface panel 290 provides one or more switches 297 and the display 238. Alternatively, the cassette 121 may couple to the saddle 283 by clipping, friction fitting, vacuum fitting, or other means.

### Clog Detection in Thrombectomy Systems

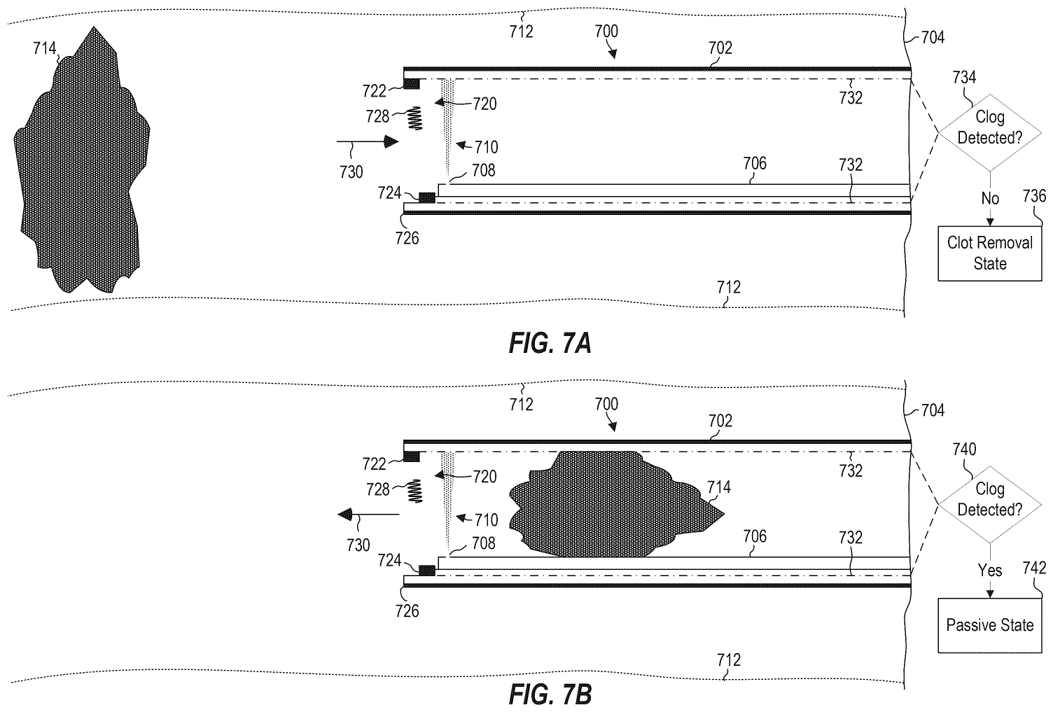

FIGS. 7A and 7B illustrate conceptual representations of components of a thrombectomy system 700. FIGS. 7A and 7B depict a distal region of an aspiration catheter 702 of the thrombectomy system 700 (break lines 704 separate the distal region of the aspiration catheter 702 from other regions thereof and from other components of the thrombectomy system 700). The distal region of an aspiration catheter 702 can comprise the distalmost 20 cm, 19 cm, 18 cm, 17 cm, 16 cm, 15 cm, 14 cm, 13 cm, 12 cm, 11 cm, 10 cm, 9 cm, 8 cm, 7 cm, 6 cm, 5 cm, 4 cm, 3 cm, 2 cm, or 1 cm of the aspiration catheter 702.

The aspiration catheter 702 of FIGS. 7A and 7B includes a supply lumen 706 with an orifice 708 that forms a fluid jet. The orifice 708 is configured to release a high-pressure saline jet 710 at or near a distal opening of the aspiration catheter 702 (e.g., as controlled by an SDU of the thrombectomy system 700).

The aspiration catheter 702 of FIGS. 7A and 7B is configured for advancement through vasculature of a subject (e.g., a human patient) to facilitate clot removal therefrom. FIGS. 7A and 7B conceptually depict vessel walls 712 through which the aspiration catheter 702 may advance and from which a clot 714 may be removed. For instance, after advancing into engagement with or proximity to the clot 714, a clot removal state of the thrombectomy system 700 may be activated. The clot removal state may cause the saline jet 710 to macerate the clot 714 and may cause negative pressure to be applied at the distal opening of the aspiration catheter 702 (e.g., by operation of one or more vacuum pumps/motors of the thrombectomy system 700) to aspirate the clot 714 and/or fragments thereof from the subject vasculature.

As noted above, while the clot removal state is active, the aspiration catheter 702 can sometimes become clogged with clot material. Users may experience difficulty in determining whether the aspiration catheter 702 has become clogged during a clot removal operation. Accordingly, the thrombectomy system 700 of FIGS. 7A and 7B includes a flow direction sensor 720. In the example of FIGS. 7A and 7B, the flow direction sensor 720 is implemented as an ultrasonic flow meter that includes ultrasonic transducers 722 and 724, which are positioned proximate to the fluid jet (formed by the orifice 708 and the supply lumen 706) at the distal end 726 of the aspiration catheter 702. FIGS. 7A and 7B depict the ultrasonic transducers 722 and 724 positioned on an inner wall of the distal opening of the aspiration catheter 702, with the ultrasonic transducers 722 and 724 being offset from one another about the inner wall of the distal opening by about 180° and being offset from one another in the distal-proximal direction (e.g., with ultrasonic transducer 722 being positioned distal to ultrasonic transducer 724 along the aspiration catheter 702). Other positional configurations of the ultrasonic transducers 722 and 724 on the aspiration catheter 702 are within the scope of the present disclosure (e.g., being offset about the catheter wall by 0°-360°, being positioned to rely on reflection of the ultrasonic signals 728 off of catheter sidewalls, omitting or having a different or distal-proximal offset configuration, being affixed to an exterior of the aspiration catheter 702 or embedded within sidewalls of the aspiration catheter 702, etc.). Although only two ultrasonic transducers 722 and 724 are shown in the example of FIGS. 7A and 7B, other quantities of ultrasonic transducers are within the scope of the present disclosure. Furthermore, although FIGS. 7A and 7B show the ultrasonic transducers 722 and 724 positioned distal to the orifice 708, other configurations are possible, such as positioning of one or both of the ultrasonic transducers 722 and/or 724 proximal to the orifice 708.

In the example of FIG. 7A, the ultrasonic transducers 722 and 724 are configured to emit ultrasonic signals 728 toward one another and detect incident ultrasonic signals 728. Ultrasonic emissions can be performed from ultrasonic transducer 722 to ultrasonic transducer 724 and vice-versa while a clot removal state is active. The ultrasonic transducers 722 and 724 can be connected to cabling 732 or other communication means that extend proximally from the ultrasonic transducers 722 and 724 along the aspiration catheter 702. The cabling 732 may facilitate signal transmission/communication between the flow direction sensor 720 and other control systems of the thrombectomy system 700.

During clot removal, the emission and detection of ultrasonic signals 728 between ultrasonic transducer 722 and ultrasonic transducer 724 can be used to determine whether the aspiration catheter 702 is clogged (e.g., with clot material). The flow of fluid through the distal region of the aspiration catheter 702 influences the time it takes for an ultrasonic signal 728 emitted by one ultrasonic transducer to traverse the interior of the aspiration catheter 702 to reach the other ultrasonic transducer. For instance, because of the distal-proximal offset of the ultrasonic transducers 722 and 724, fluid flow through the aspiration catheter 702 in the proximal direction (indicated by arrow 730 in FIG. 7A) can cause an ultrasonic signal 728 emitted by ultrasonic transducer 722 to reach ultrasonic transducer 724 in less time than it would take for an ultrasonic signal 728 emitted by ultrasonic transducer 724 to reach ultrasonic transducer 722. In contrast, fluid flow in through the aspiration catheter 702 in the distal direction (indicated by arrow 730 in FIG. 7B) can cause an ultrasonic signal 728 emitted by ultrasonic transducer 724 to reach ultrasonic transducer 722 in less time than it would take for an ultrasonic signal 728 emitted by ultrasonic transducer 722 to reach ultrasonic transducer 724. By analyzing and/or comparing the time-of-flight or transit time of the ultrasonic signals 728 emitted in the different directions (e.g., toward ultrasonic transducer 722 and toward ultrasonic transducer 724) the flow direction of fluid through the aspiration catheter 702 can be determined. For instance, a system can analyze the emission and detection times of the ultrasonic signals 728 between the ultrasonic transducers 722 and 724 in the different directions and determine whether flow through the distal region of the aspiration catheter 702 is in the proximal direction (indicated by arrow 730 in FIG. 7A) or the distal direction (indicated by arrow 730 in FIG. 7B). Other techniques may be employed to determine flow direction with ultrasonic transducers, such as Doppler shift techniques.

FIG. 7A depicts an instance in which a clot removal state is active, where a high-pressure saline jet 710 is formed and negative pressure is applied to the aspiration catheter 702 (e.g., via vacuum components of the thrombectomy system 700). In the example of FIG. 7A, the aspiration catheter 702 is not clogged (e.g., the aspiration catheter 702 has not reached the clot 714 and is not clogged by the clot 714). Accordingly, the negative pressure applied to the aspiration catheter 702 causes suction of fluid (e.g., saline, blood, clot material) into the distal end 726 and/or through the aspiration catheter 702 in a proximal direction (indicated by arrow 730 in FIG. 7A). While the clot removal state is active, ultrasonic signals 728 may be emitted from ultrasonic transducer 722 toward 724, and vice-versa. The thrombectomy system 700 can analyze and/or compare the emission and detection times of the ultrasonic signals 728 between the ultrasonic transducers 722 and 724 in the different directions and determine that the time-of-flight is less for ultrasonic signals 728 propagating from ultrasonic transducer 722 toward ultrasonic transducer 724. Such information can indicate that fluid is flowing through the distal region of the aspiration catheter 702 in the proximal direction (indicated by arrow 730 in FIG. 7A).

In some implementations, the thrombectomy system 700 may selectively remain in the clot removal state in response to determining that the emission/detection information associated with the flow direction sensor 720 indicates that fluid is flowing at the distal region of the aspiration catheter 702 in the proximal direction (indicated by arrow 730 in FIG. 7A). Fluid flow in the proximal direction (e.g., with sufficient velocity) can indicate that the aspiration catheter 702 is not clogged (as indicated in FIG. 7A by decision block 734 and the “No” arrow extending therefrom toward action block 736). By remaining in the clot removal state, aspiration components of the thrombectomy system 700 (e.g., the vacuum pump/motor, SDU, etc.) can continue to generate negative pressure and provide a saline jet 710 at the distal region of the aspiration catheter 702 to facilitate clot aspiration and/or maceration.

FIG. 7B also depicts an instance in which the clot removal state is active. In the example of FIG. 7B, the aspiration catheter 702 has become clogged (e.g., the aspiration catheter reached the clot 714, though the high-pressure saline jet 710 failed to macerate the clot 714, resulting in clogging of the clot 714 within the distal region of the aspiration catheter 702). Accordingly, the negative pressure applied to the aspiration catheter 702 fails to cause suction of fluid (e.g., saline, blood, clot material) through the aspiration catheter 702 in the proximal direction. Instead, continued jetting of fluid from the orifice 708 pursuant to the clot removal state causes a flow of fluid in the distal direction (indicated by arrow 730 in FIG. 7B) in at least part of the distal region of the aspiration catheter 702. While the clot removal state is active, ultrasonic signals 728 may be emitted from ultrasonic transducer 722 toward 724, and vice-versa. The thrombectomy system 700 can analyze and/or compare the emission and detection times of the ultrasonic signals 728 between the ultrasonic transducers 722 and 724 in the different directions and determine that the time-of-flight is less for ultrasonic signals 728 propagating from ultrasonic transducer 724 toward ultrasonic transducer 722 (or determine that the time-of-flight is within a threshold similarity for both directions, or determine that the time-of-flight fails to satisfy one or more conditions for ultrasonic signals traveling from ultrasonic transducer 722 to ultrasonic transducer 724). Such information can indicate that fluid is flowing through at least part of the distal region of the aspiration catheter 702 in the distal direction (indicated by arrow 730 in FIG. 7B).

In some implementations, the thrombectomy system 700 may selectively deactivate the clot removal state and/or enter a passive state in response to determining that the emission/detection information associated with the flow direction sensor 720 indicates that fluid is flowing at the distal region of the aspiration catheter 702 in the distal direction (indicated by arrow 730 in FIG. 7B). Fluid flow in the distal direction can indicate that the aspiration catheter 702 is clogged (as indicated in FIG. 7B by decision block 740 and the “Yes” arrow extending therefrom toward action block 742). The passive state may comprise a state in which aspiration components of the thrombectomy system 700 (e.g., the vacuum pump/motor, SDU, etc.) refrain from producing the high-pressure saline jet 710, refrain from generating negative pressure at the distal region of the aspiration catheter 702, or only generate a low level of negative pressure at the distal region (e.g., pursuant to a low-level aspiration state). Additionally, or alternatively, the thrombectomy system 700 may present an alert on a user interface of the thrombectomy system 700 in response to determining that the emission/detection information associated with the flow direction sensor 720 indicates that fluid is flowing at the distal region of the aspiration catheter 702 in the distal direction. An alert may comprise an indication that the aspiration catheter 702 of the thrombectomy system 700 is clogged (or potentially clogged), a directive to deactivate the clot removal state (or enter the passive state), and/or other content.

Although FIGS. 7A and 7B focus on an example in which a flow direction sensor 720 comprises an ultrasonic flow meter, other types of devices may be utilized to determine the direction of fluid flow at a distal region of an aspiration catheter 702 (indicating the clog state of the aspiration catheter).

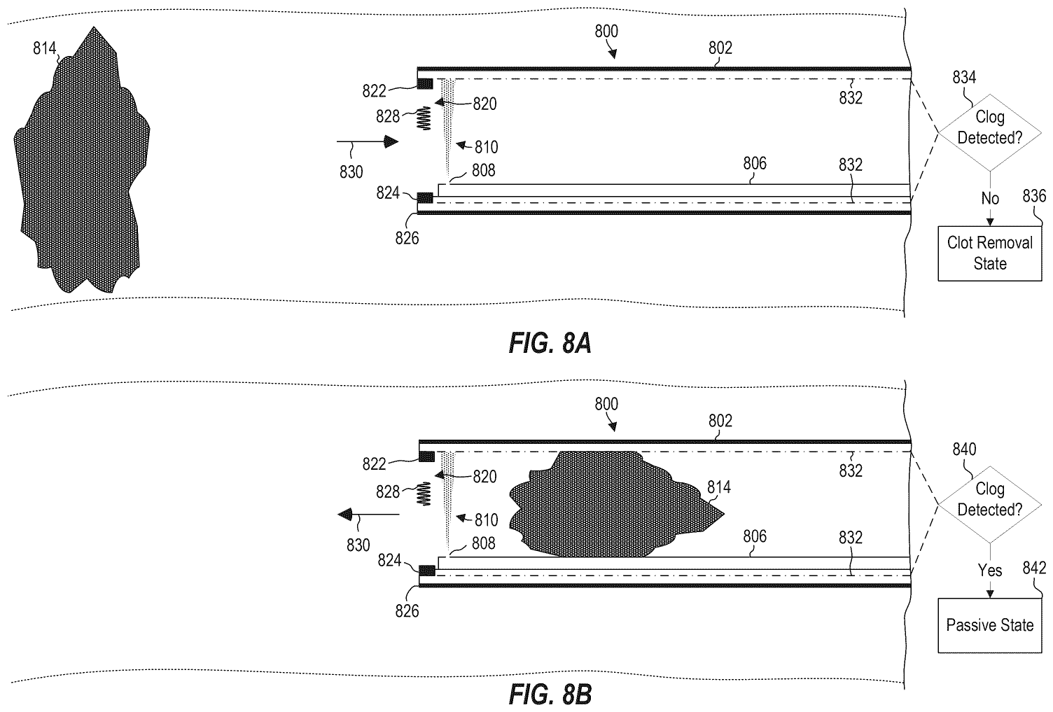

As one example, FIGS. 8A and 8B illustrate conceptual representations of a thrombectomy system 800 that implements a flow direction sensor 820 in the form of an optical flow sensor that includes a light emitter 822 and a light detector 824. (e.g., positioned similar to the potential positioning of ultrasonic transducers 722 and 724 described hereinabove). The thrombectomy system 800 includes components similar to thrombectomy system 700, such as an aspiration catheter 802, a supply lumen 806 with an orifice 808 for releasing a saline jet 810, etc. FIGS. 8A and 8B illustrate the light emitter 822 and the light detector 824 positioned proximate to the fluid jet 810 (formed by the orifice 808 and the supply lumen 806) at the distal end 826 of the aspiration catheter 802. Various positionings of the light emitter 822 and the light detector 824 are within the scope of the present disclosure (e.g., similar to the positionings of the ultrasonic transducers 722 and 724 described hereinabove). Any quantity or combination of light emitters and light detectors can be included in the thrombectomy system 800. The light emitter 822 and the light detector 824 of FIGS. 8A and 8B are connected to cabling 832 to facilitate control of the light emitter 822 and the light detector 824 and/or communication with other components of the thrombectomy system 800.

In the example of FIGS. 8A and 8B, the light emitter 822 (e.g., a laser or other light source) emits light 828 toward the light detector 824 (e.g., a photodetector) to enable the light detector 824 to detect the emitted light 828 after interaction with particles within the fluid flowing through the aspiration catheter 802 (e.g., during operation of the clot removal state of the thrombectomy system 800). The particles within the fluid flowing through the aspiration catheter 802 can cause a frequency shift in the light 828 as it propagates toward the light detector 824. The frequency shift in the light 828 detected by the light detector 824 can indicate the flow rate of the fluid, and the sign of the frequency shift can indicate the direction of saline fluid flow (e.g., via Doppler shift analysis). The determined direction of saline fluid flow based on the flow direction sensor 820 can be used to infer the clog state of the aspiration catheter 802, which may trigger different actions.

For example, FIG. 8A shows the thrombectomy system 800 operating in a clot removal state without a clog present in the aspiration catheter 802. Fluid thus freely moves through the aspiration catheter 802 in a proximal direction (indicated by arrow 830 in FIG. 8A). The flow direction sensor 820 can measure the frequency shifts in the light 828 emitted by the light emitter 822 and detected by the light detector 824 caused by interactions between the light 828 and the fluid traveling through the aspiration catheter 802 in the proximal direction. The thrombectomy system 800 may selectively remain in the clot removal state in response to determining that the frequency shift information associated with the flow direction sensor 820 indicates that fluid is flowing at the distal region of the aspiration catheter 802 in the proximal direction (indicated by arrow 830 in FIG. 8A). Fluid flow in the proximal direction (e.g., with sufficient velocity) can indicate that the aspiration catheter 802 is not clogged (as indicated in FIG. 8A by decision block 834 and the “No” arrow extending therefrom toward action block 836).

FIG. 8B also depicts an instance in which the clot removal state is active. In the example of FIG. 8B, the aspiration catheter 802 has become clogged (e.g., the aspiration catheter reached the clot 814, though the high-pressure saline jet 810 failed to macerate the clot 814, resulting in clogging of the clot 814 within the distal region of the aspiration catheter 802). Accordingly, the negative pressure applied to the aspiration catheter 802 fails to cause suction of fluid (e.g., saline, blood, clot material) through the aspiration catheter 802 in the proximal direction. Instead, continued jetting of fluid from the orifice 808 pursuant to the clot removal state causes a flow of fluid in the distal direction (indicated by arrow 830 in FIG. 8B) in at least part of the distal region of the aspiration catheter 802. The flow direction sensor 820 can measure the frequency shifts in the light 828 emitted by the light emitter 822 and detected by the light detector 824 caused by interactions between the light 828 and the fluid traveling through the aspiration catheter 802 in the distal direction. The thrombectomy system 800 may selectively deactivate the clot removal state and/or enter a passive state in response to determining that the frequency shift information associated with the flow direction sensor 820 indicates that fluid is flowing at the distal region of the aspiration catheter 802 in the distal direction (indicated by arrow 830 in FIG. 8B). Fluid flow in the distal direction can indicate that the aspiration catheter 802 is clogged (as indicated in FIG. 8B by decision block 840 and the “Yes” arrow extending therefrom toward action block 842).

Other than Doppler shift techniques, the flow direction sensor 820 can utilize other types of optical detection techniques to facilitate clog state detection for the thrombectomy system 800. For example, in some implementations, the light 828 emitted by the light emitter 822 and detected by the light detector 824 is used to facilitate measurement of analytes present in blood (“blood analytes”). The concentration of blood analytes in the fluid passing through the distal region of the aspiration catheter 802 can indicate the flow direction of the fluid.

For example, during operation of the clot removal state in the absence of a catheter clog, the blood analyte measurements of the fluid passing through the propagation path of the light 828 between the light emitter 822 and the light detector 824 (positioned as shown in FIG. 8A) can be expected to predominantly comprise blood (partial mixing with saline from the saline jet 810 can occur). Thus, the blood analyte measurements of the passing fluid can be expected to approximate or approach normal levels (e.g., typical or standard ranges of values established based on blood analysis of healthy populations) or modified levels that account for partial mixing of the saline from the saline jet 810 with the blood. The expected amount of mixing between blood and saline for fluid passing the flow direction sensor 820 during operation of the clot removal state without a catheter clog can depend on the positioning of the flow direction sensor 820 (e.g., greater mixing can be expected with the flow direction sensor 820 positioned proximal to the saline jet 810, rather than distal to the saline jet 810 as shown in FIG. 8A).

Conversely, during operation of the clot removal state in the presence of a catheter clog, the blood analyte measurements of fluid passing through the propagation path of the light 828 between the light emitter 822 and the light detector 824 (positioned as shown in FIG. 8B) are expected to be drastically lower than normal levels due to mitigation of the blood entering the aspiration catheter 802, resulting in dominance of the saline from the saline jet 810 in the fluid passing through the propagation path.

Thus, analyte concentration in the fluid passing through the propagation path between the light emitter 822 and the light detector 824 can indicate whether a clog is present in the aspiration catheter 802. When the measured analyte concentration satisfies one or more conditions (e.g., a threshold), the system can determine that no catheter clog is present and remain in the clot removal state (e.g., according to decision block 834 and action block 836 of FIG. 8A). When the measured analyte concentration fails to satisfy the condition(s) (e.g., the threshold), the system can determine that a catheter clog is present and enter a passive state (e.g., according to decision block 840 and action block 842 of FIG. 8B). In some instances, a flow direction sensor 820 includes multiple temperature sensors (e.g., positioned proximal and/or distal to the fluid jet 810), and data collected from the multiple temperature sensors is aggregated to determine clog state for the thrombectomy system 800.

Various analytes can be measured via the light emitter 822 and the light detector 824 to facilitate clog state detection of the thrombectomy system 800 as described above. For example, hemoglobin is associated with particular absorption features within a range of about 535 nm to about 580 nm, such as absorption peaks for oxygenated hemoglobin of about 540 nm and 575-576 nm and an absorption peak for deoxygenated hemoglobin within a range of about 555 nm to about 560 nm. Thus, in some implementations, the light emitter 822 is configured to emit light 828 of one or more known wavelengths that include absorption peaks associated with hemoglobin, and attenuation coefficients of the light 828 detected by the light detector 824 can be analyzed to provide an estimated hemoglobin concentration in the fluid traversed by the light 828. As noted above, the estimated hemoglobin concentration can indicate whether or not a catheter clog is present and can trigger different actions.

Additional or alternative blood analytes associated with known light absorption or emission characteristics can be measured via the flow direction sensor 820 to facilitate determination of catheter clog state for the thrombectomy system 800 (e.g., bilirubin, porphyrins, and/or others). In some implementations, a flow direction sensor can utilize non-optical techniques to facilitate analyte measurement indicating catheter clog state detection, such as electrode-based, electromechanical sensors-based, and/or enzyme-based methods (e.g., to measure blood glucose levels, lactate levels, cholesterol levels, urea levels, creatinine levels, pH levels, potassium levels, sodium levels, carbon dioxide levels, bilirubin levels, and/or others).

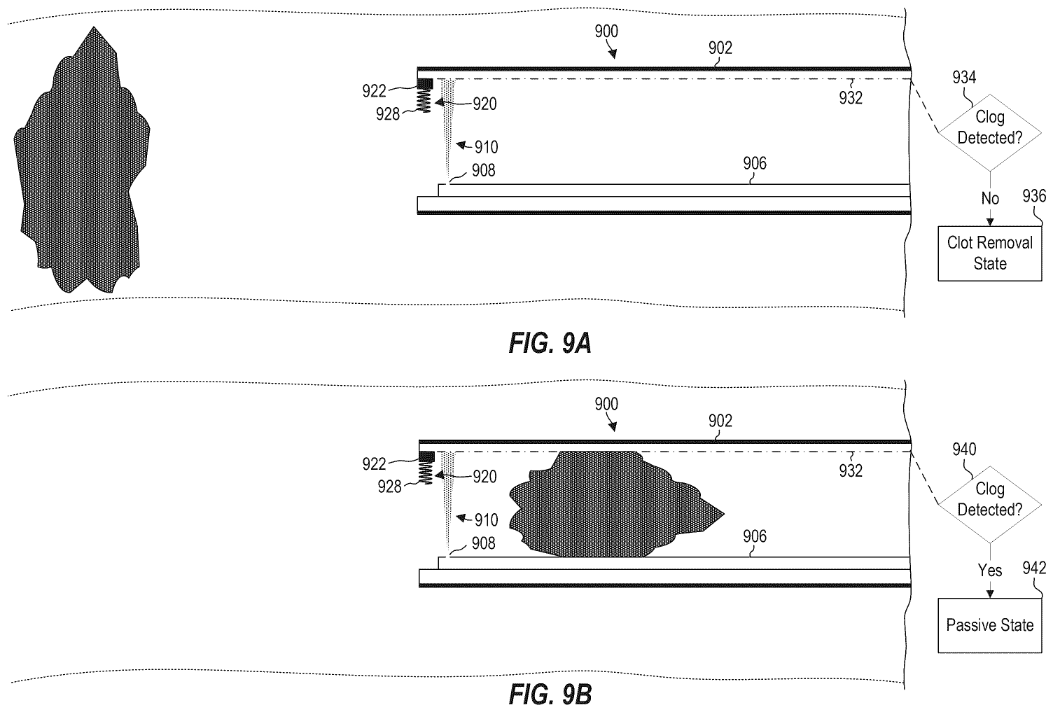

FIGS. 9A and 9B provide an additional example of an optical-based flow direction sensor 920 similar to the flow direction sensor 820 discussed hereinabove with reference to FIGS. 8A and 8B. The optical-based flow direction sensor 920 is implemented on a thrombectomy system 900 that includes components similar to thrombectomy system 700, such as an aspiration catheter 902, a supply lumen 906 with an orifice 908 for releasing a saline jet 910, etc. The optical-based flow direction sensor 920 comprises a photonic device 922 that operates as both a light emitter (able to perform the functions of the light emitter 822 discussed hereinabove) and a light detector (able to perform the functions of the light detector 824 discussed hereinabove). During operation of the clot removal state, the photonic device 922 of FIGS. 9A and 9B is configured to emit light 928, which is at least partially reflected back toward the photonic device 922 by interaction with particles of the fluid passing through the aspiration catheter 902. The reflected light 928 can be processed by the thrombectomy system 900 to determine whether a catheter clog is present. For example, the reflected light 928 can be processed to estimate analyte levels within the fluid passing the optical-based flow direction sensor 920, which can indicate the flow direction of fluid at the distal region of the aspiration catheter 902, which can indicate whether a catheter clog is present. As an illustrative example, to facilitate detection of hemoglobin concentration, the photonic device 922 can comprise a central illumination core for emitting light. The central illumination core can be surrounded by collection fibers, advantageously enabling signal emission and collection to occur at a single location. The photonic device 922 can be positioned distal to the saline jet 910 (as shown in FIGS. 9A and 9B) or proximal to the saline jet 910. The photonic device 922 can be configured to emit light including various wavelengths, such as one or more ranges of wavelengths that include about 500 nm, about 509 nm, about 520 nm, and/or about 529 nm. The photonic device 922 can detect diffuse reflections of the light to facilitate analysis of reflectance ratios to determine hemoglobin concentration. For instance, contrast in the diffuse reflectance ratio of 529/500 nm can be caused by the absorption of hemoglobin, enabling the measured 529/500 nm diffuse reflectance ratio to be used to measure hemoglobin concentration. A baseline ratio (e.g., 520/509 nm) can be subtracted from the 529/500 nm diffuse reflectance ratio to address background noise in the measurement signal. The measurement signal can be analyzed overtime to detect a reduction in hemoglobin concentration, which can indicate the presence of a clog in the aspiration catheter 902.

If a catheter clog is not present, the thrombectomy system 900 can remain in the clot removal state (indicated by the “No” arrow extending from decision block 934 toward action block 936 in FIG. 9A). If a catheter clog is present, the thrombectomy system 900 can enter a passive state (indicated by the “Yes” arrow extending from decision block 940 toward action block 942 in FIG. 9B).

The photonic device 922 of FIGS. 9A and 9B can be positioned distal to or proximal to the saline jet 910. Any quantity of photonic device 922 can be included in the thrombectomy system 900. The photonic device 922 is connected to cabling 932 to facilitate control of the photonic device 922 and/or communication with other components of the thrombectomy system 900.

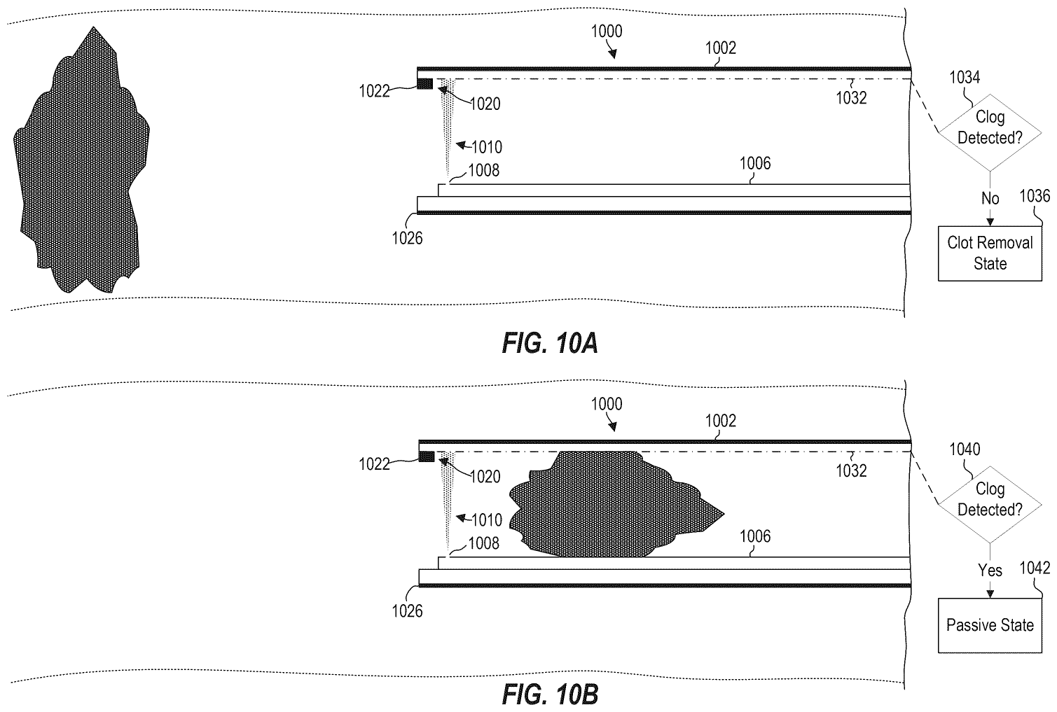

Another example technique for detecting fluid flow direction associated with an aspiration catheter of a thrombectomy system utilizes temperature measurements. FIGS. 10A and 10B illustrate conceptual representations of a thrombectomy system 1000 that implements a flow direction sensor 1020 in the form of a temperature sensor 1022. FIGS. 10A and 10B illustrate the temperature sensor 1022 positioned proximate to the fluid jet 1010 (formed by the orifice 1008 and the supply lumen 1006) at the distal end 1026 of the aspiration catheter 1002. Various positionings of the temperature sensor 1022 are within the scope of the present disclosure, such as positionings distal to the fluid jet 1010 and/or positionings proximal to the fluid jet 1010. Any quantity of temperature sensors can be included in the thrombectomy system 1000. The temperature sensor 1022 of FIGS. 10A and 10B is connected to cabling 1032 to facilitate control of the temperature sensor 1022 and/or communication with other components of the thrombectomy system 1000.

The temperature sensor 1022 of FIGS. 10A and 10B can comprise a micro-electromechanical system (MEMS) thermocouple for which changes in temperature cause changes in voltage, enabling measurement of temperature and/or change in temperature. The temperature of the fluid passing the temperature sensor 1022 can indicate the direction of fluid flow, which can indicate whether a clog is present in the aspiration catheter 1002. For example, during operation of the clot removal state in the absence of a catheter clog, the temperature of the fluid passing the temperature sensor 1022 (positioned as shown in FIG. 10A) can be expected to predominantly comprise blood (partial mixing with saline from the fluid jet 1010 can occur). Thus, the temperature of the fluid passing the temperature sensor 1022 can be expected to approximate or approach normal levels (e.g., about 37° C.) or modified levels that account for partial mixing of the saline from the saline jet 1010 with the blood. The expected amount of mixing between blood and saline for fluid passing the flow direction sensor 1020 during operation of the clot removal state without a catheter clog can depend on the positioning of the flow direction sensor 1020 (e.g., greater mixing can be expected with the flow direction sensor 1020 positioned proximal to the saline jet 1010, rather than distal to the saline jet 1010 as shown in FIG. 10A).

Conversely, during operation of the clot removal state in the presence of a catheter clog, the temperature of fluid passing the temperature sensor 1022 (positioned as shown in FIG. 10B) is expected to be drastically lower than normal levels (e.g., room temperature, or about 25° C., or another temperature value or range less than about 37°) due to mitigation of the blood entering the aspiration catheter 1002, resulting in dominance of the (colder) saline from the saline jet 1010 in the fluid passing the temperature sensor 1022.

Thus, the temperature of the fluid passing the temperature sensor 1022 can indicate whether a clog is present in the aspiration catheter 1002. When the measured temperature satisfies one or more conditions (e.g., a threshold, such as about 37° C.), the system can determine that no catheter clog is present and remain in the clot removal state (e.g., according to decision block 1034 and action block 1036 of FIG. 10A). When the measured temperature fails to satisfy the condition(s) (e.g., the threshold), the system can determine that a catheter clog is present and enter a passive state (e.g., according to decision block 1040 and action block 1042 of FIG. 10B). In some instances, a flow direction sensor 1020 includes multiple temperature sensors (e.g., positioned proximal and/or distal to the fluid jet 1010), and data collected from the multiple temperature sensors is aggregated to determine clog state for the thrombectomy system 1000.

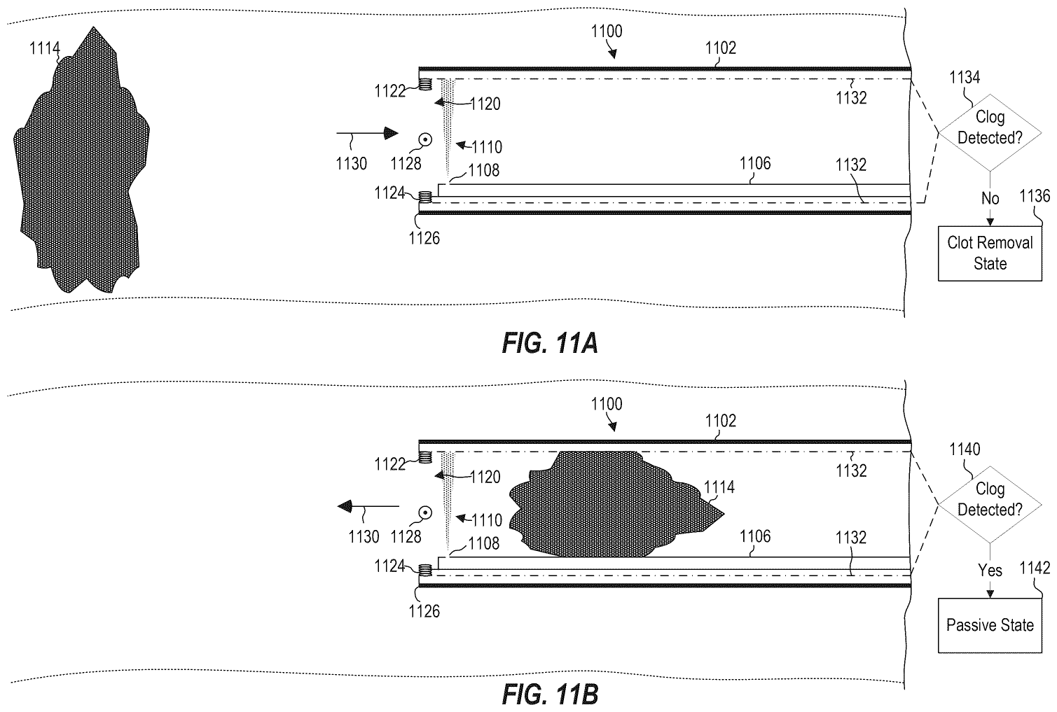

As another example, FIGS. 11A and 11B illustrate conceptual representations of a thrombectomy system 1100 that implements a flow direction sensor 1120 in the form of an electromagnetic flow meter that includes coils 1122 and 1124 and electrodes 1128 (only one electrode is illustrated in FIGS. 11A and 11B due to the cross-sectional representation of the thrombectomy system 1100). The thrombectomy system 1100 includes components similar to thrombectomy system 700, such as an aspiration catheter 1102, a supply lumen 1106 with an orifice 1108 for releasing a saline jet 1110, etc.

In the example of FIGS. 11A and 11B, coils 1122 and 1124 and electrodes 1128 are positioned proximate to the fluid jet (formed by orifice 1108 and the supply lumen 1106) at the distal end 1126 of the aspiration catheter 1102. FIGS. 11A and 11B depict the coils 1122 and 1124 on opposing sides of the aspiration catheter 1102 to form a magnetic field across the cavity of the aspiration catheter 1102. The electrodes 1128 are positioned on other portions of the sidewall of the aspiration catheter 1102. The positional configuration of the coils 1122 and 1124 and the electrodes 1128 of FIGS. 11A and 11B is provided by way of example only and can be varied in implementation (e.g., at least part of the flow direction sensor 1120 can be positioned proximal to the orifice 1108 that forms the saline jet 1110). Each of the coils 1122 and 1124 and the electrodes 1128 can be connected to cabling 1132 to facilitate communication with other components of the thrombectomy system 1100.

As fluid flows through the aspiration catheter 1102 and the magnetic field formed by the coils 1122 and 1124, a voltage signal (e.g., electromotive force (EMF)) may be induced across the electrodes 1128 (e.g., perpendicular to both the direction of the magnetic field and the direction of fluid flow). The induced voltage can indicate the flow rate of the fluid passing through the aspiration catheter 1102, and the polarity of the induced voltage can indicate the direction of the fluid passing through the aspiration catheter 1102. Thus, while operating in a clot removal state, the flow direction sensor 1120 can indicate the direction of flow through the distal region of the aspiration catheter 1102, which can indicate whether the aspiration catheter 1102 is clogged (e.g., by clot material).

For example, FIG. 11A shows the thrombectomy system 1100 operating in a clot removal state without a clog present in the aspiration catheter 1102. Fluid thus freely moves through the aspiration catheter 1102 in a proximal direction (indicated by arrow 1130 in FIG. 11A). The flow direction sensor 1120 can measure the polarity of the voltage induced in the electrodes 1128 by fluid traveling through the aspiration catheter 1102 across the magnetic field formed by the coils 1122 and 1124 to determine that the fluid is flowing in the proximal direction. The thrombectomy system 1100 may selectively remain in the clot removal state in response to determining that the induced voltage polarity information associated with the flow direction sensor 1120 indicates that fluid is flowing at the distal region of the aspiration catheter 1102 in the proximal direction (indicated by arrow 1130 in FIG. 11A). Fluid flow in the proximal direction (e.g., with sufficient velocity) can indicate that the aspiration catheter 1102 is not clogged (as indicated in FIG. 11A by decision block 1134 and the “No” arrow extending therefrom toward action block 1136).

FIG. 11B also depicts an instance in which the clot removal state is active. In the example of FIG. 11B, the aspiration catheter 1102 has become clogged (e.g., the aspiration catheter reached the clot 1114, though the high-pressure saline jet 1110 failed to macerate the clot 1114, resulting in clogging of the clot 1114 within the distal region of the aspiration catheter 1102). Accordingly, the negative pressure applied to the aspiration catheter 1102 fails to cause suction of fluid (e.g., saline, blood, clot material) through the aspiration catheter 1102 in the proximal direction. Instead, continued jetting of fluid from the orifice 1108 pursuant to the clot removal state causes a flow of fluid in the distal direction (indicated by arrow 1130 in FIG. 11B) in at least part of the distal region of the aspiration catheter 1102. The flow direction sensor 1120 can measure the polarity of the voltage induced in the electrodes 1128 by fluid traveling through the aspiration catheter 1102 across the magnetic field formed by the coils 1122 and 1124 to determine that the fluid is flowing in the distal direction. The thrombectomy system 1100 may selectively deactivate the clot removal state and/or enter a passive state in response to determining that the induced voltage polarity information associated with the flow direction sensor 1120 indicates that fluid is flowing at the distal region of the aspiration catheter 1102 in the distal direction (indicated by arrow 1130 in FIG. 11B). Fluid flow in the distal direction can indicate that the aspiration catheter 1102 is clogged (as indicated in FIG. 11B by decision block 1140 and the “Yes” arrow extending therefrom toward action block 1142).

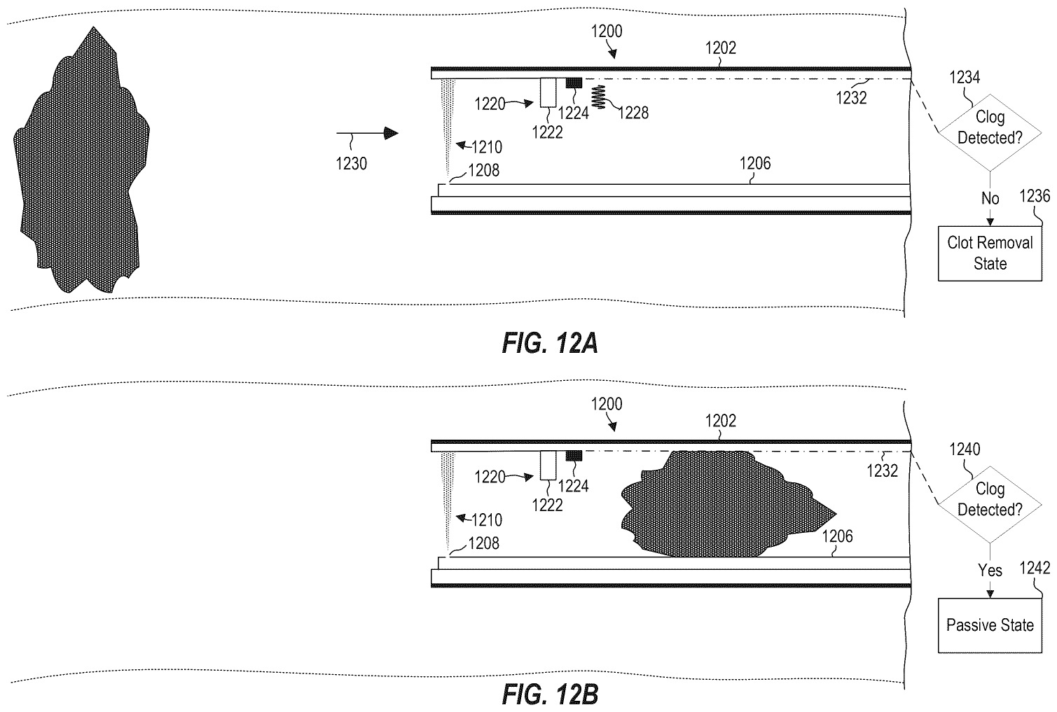

In some implementations, flow sensors that determine flow rate, regardless of flow direction, may be utilized to estimate/determine the clog state of an aspiration catheter of a thrombectomy system. For instance, FIGS. 12A and 12B illustrate a thrombectomy system 1200 that includes a flow sensor 1220 implemented as a vortex shedding flow meter that includes a bluff body 1222 and a pressure sensor 1224 (connected to cabling 1232). The thrombectomy system 1200 includes components similar to thrombectomy system 700, such as an aspiration catheter 1202, a supply lumen 1206 with an orifice 1208 for releasing a saline jet 1210, etc. The flow sensor 1220 of FIGS. 12A and 12B is illustrated as being positioned proximal to the orifice 1208 that forms the fluid jet 1210 (though other configurations may be used). The flow sensor 1220 is adapted to detect the flow rate of fluid within the aspiration lumen of the aspiration catheter 1202 during operation of a clot removal state. Characteristics of the detected flow rate can indicate whether the aspiration catheter 1202 is clogged.

As fluid flows past the bluff body 1222 within the aspiration catheter 1202, vortices 1228 are alternately shed on either side of the bluff body 1222. The shedding frequency is proportional to the flow rate of the fluid. The pressure sensor 1224 can detect vortices 1228 shed via the bluff body 1222 and can therefore enable determination of the vortex shedding frequency, providing an indication of the flow rate of the fluid within the aspiration catheter 1202.

When the measured flow rate satisfies one or more conditions, such as exceeding threshold flow rate levels, the thrombectomy system 1200 can determine that no clog is present in the aspiration catheter 1202. This is shown in FIG. 12A, which illustrates the thrombectomy system 1200 operating in a clot removal state without a clog present in the aspiration catheter 1202. Fluid thus freely moves through the aspiration catheter 1202 in a proximal direction (indicated by arrow 1230 in FIG. 12A), allowing the flow sensor 1220 to measure a flow rate that satisfies conditions to indicate that no clog is present, which can cause the thrombectomy system 1200 to remain in the clot removal state (as indicated in FIG. 12A by decision block 1234 and the “No” arrow extending therefrom toward action block 1236).

When the measured flow rate fails to satisfy the condition(s), the thrombectomy system 1200 can determine that a clog is present in the aspiration catheter 1202. This is shown in FIG. 12B, which illustrates the thrombectomy system 1200 operating in the clot removal state with a clog present in the aspiration catheter 1202. Accordingly, the negative pressure applied to the aspiration catheter 1202 fails to cause a high magnitude of suction of fluid (e.g., saline, blood, clot material) through the aspiration catheter 1202 in the proximal direction. The flow sensor 1220 thus measures a flow rate that indicates that a clog is present, which can cause the thrombectomy system 1200 to enter a passive state (as indicated in FIG. 12B by decision block 1240 and the “Yes” arrow extending therefrom toward action block 1242).

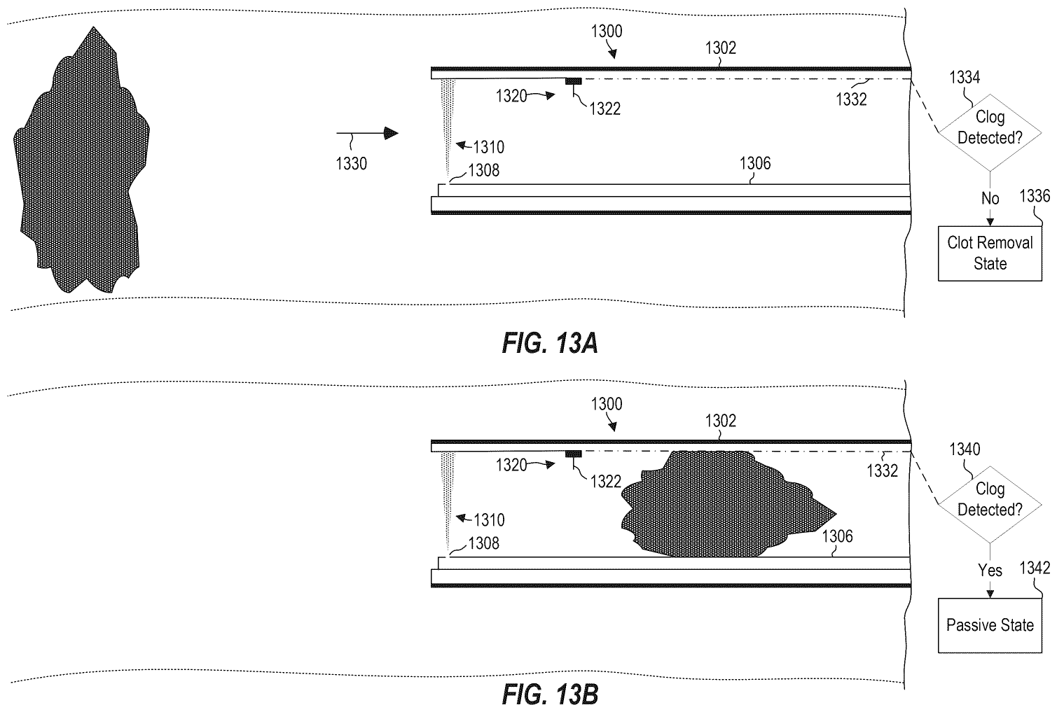

As another example, FIGS. 13A and 13B illustrate a thrombectomy system 1300 that includes a flow sensor 1320 implemented as a thermal flow meter that includes a plurality of temperature sensors 1322 (connected to cabling 1332). The thrombectomy system 1300 includes components similar to thrombectomy system 700, such as an aspiration catheter 1302, a supply lumen 1306 with an orifice 1308 for releasing a saline jet 1310, etc. The flow sensor 1320 of FIGS. 13A and 13B is illustrated as being positioned proximal to the orifice 1308 that forms the fluid jet 1310 (though other configurations may be used).

The temperature sensors 1322 can include a first temperature sensor that measures the temperature of the fluid within the aspiration catheter 1302 and a second temperature sensor that is actively heated via an electrical current to maintain a predefined temperature offset between the first and second temperature sensors. The first and second temperature sensors can be placed within close proximity to one another. Fluid flowing past the plurality of temperature sensors 1322 exhibits a cooling effect on the plurality of temperature sensors 1322, which can cause changes to the electrical current applied to the second temperature sensor to maintain the predefined temperature offset. The electrical current applied to the second temperature sensor to compensate for the cooling effect brought about by fluid flow is proportional to the flow rate of fluid within the aspiration catheter 1302. Thus, the electrical current applied to the second temperature sensor can provide an indication of the flow rate of fluid within the aspiration catheter 1302. In an alternative embodiment, the electrical current applied to the second temperature sensor can be held constant, and the temperature differential between the two temperature sensors can indicate flow rate. In another alternative embodiment, a temperature sensor can be positioned proximal to an actively heated temperature sensor, which can enable the proximal temperature sensor to measure heat dissipation characteristics, which can indicate flow rate.

When the measured flow rate satisfies one or more conditions, such as exceeding threshold flow rate levels, the thrombectomy system 1300 can determine that no clog is present in the aspiration catheter 1302. This is shown in FIG. 13A, which illustrates the thrombectomy system 1300 operating in a clot removal state without a clog present in the aspiration catheter 1302. Fluid thus freely moves through the aspiration catheter 1302 in a proximal direction (indicated by arrow 1330 in FIG. 13A), allowing the flow sensor 1320 to measure a flow rate that satisfies conditions to indicate that no clog is present, which can cause the thrombectomy system 1300 to remain in the clot removal state (as indicated in FIG. 13A by decision block 1334 and the “No” arrow extending therefrom toward action block 1336).

When the measured flow rate fails to satisfy the condition(s), the thrombectomy system 1300 can determine that a clog is present in the aspiration catheter 1302. This is shown in FIG. 13B, which illustrates the thrombectomy system 1300 operating in the clot removal state with a clog present in the aspiration catheter 1302. Accordingly, the negative pressure applied to the aspiration catheter 1302 fails to cause a high magnitude of suction of fluid (e.g., saline, blood, clot material) through the aspiration catheter 1302 in the proximal direction. The flow sensor 1320 thus measures a flow rate that indicates that a clog is present, which can cause the thrombectomy system 1300 to enter a passive state (as indicated in FIG. 13B by decision block 1340 and the “Yes” arrow extending therefrom toward action block 1342).

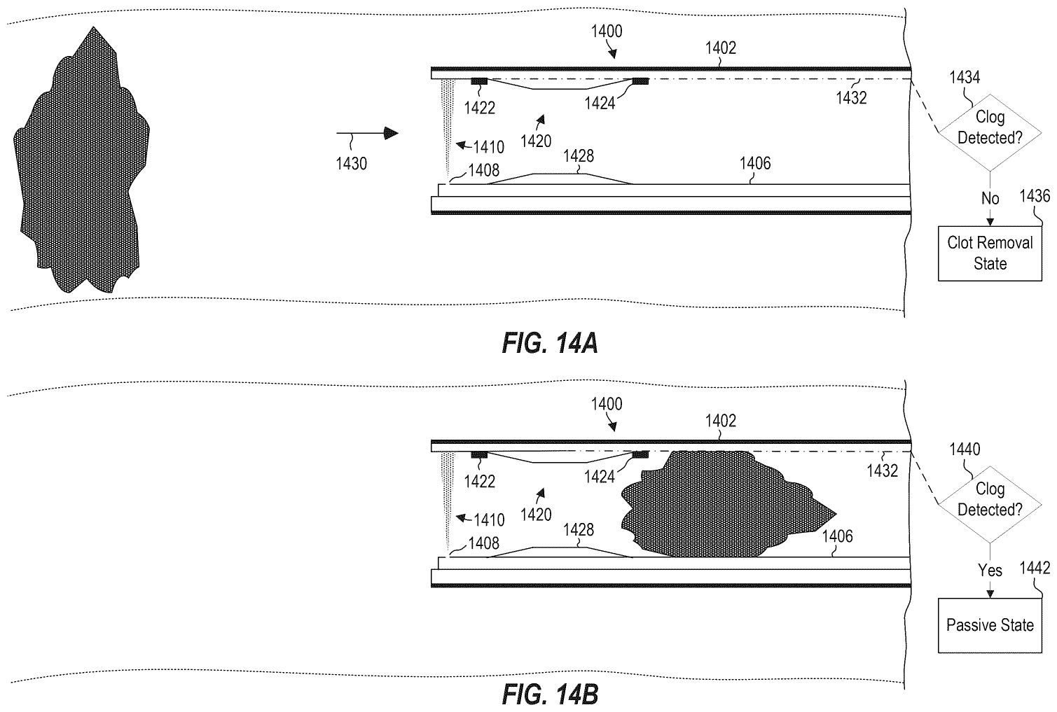

As yet another example, FIGS. 14A and 14B illustrate a thrombectomy system 1400 that includes a flow sensor 1420 implemented as a differential pressure flow meter that includes pressure sensors 1422 and 1424 (connected to cabling 1432) and a restrictive element 1428. The thrombectomy system 1400 includes components similar to thrombectomy system 700, such as an aspiration catheter 1402, a supply lumen 1406 with an orifice 1408 for releasing a saline jet 1410, etc. The flow sensor 1420 of FIGS. 14A and 14B is illustrated as being positioned proximal to the orifice 1408 that forms the fluid jet 1410 (though other configurations may be used).

As fluid flows through the restrictive element 1428, the velocity of the fluid increases, causing a pressure difference on different sides of the restrictive element 1428 (e.g., a pressure drop from the distal side to the proximal side when flow is in the proximal direction, indicated by arrow 1430 in FIG. 14A). The pressure difference is quantified by the pressure sensors 1422 and 1424, which are positioned on different sides of the restrictive element 1428. The differential pressure as measured via the pressure sensors 1422 and 1424 is proportional to the flow rate of the fluid. The differential pressure can therefore provide an indication of the flow rate of the fluid within the aspiration catheter 1402.

When the measured flow rate satisfies one or more conditions, such as exceeding threshold flow rate levels, the thrombectomy system 1400 can determine that no clog is present in the aspiration catheter 1402. This is shown in FIG. 14A, which illustrates the thrombectomy system 1400 operating in a clot removal state without a clog present in the aspiration catheter 1402. Fluid thus freely moves through the aspiration catheter 1402 in a proximal direction (indicated by arrow 1430 in FIG. 14A), allowing the flow sensor 1420 to measure a flow rate that satisfies conditions to indicate that no clog is present, which can cause the thrombectomy system 1400 to remain in the clot removal state (as indicated in FIG. 14A by decision block 1434 and the “No” arrow extending therefrom toward action block 1436).

When the measured flow rate fails to satisfy the condition(s), the thrombectomy system 1400 can determine that a clog is present in the aspiration catheter 1402. This is shown in FIG. 14B, which illustrates the thrombectomy system 1400 operating in the clot removal state with a clog present in the aspiration catheter 1402. Accordingly, the negative pressure applied to the aspiration catheter 1402 fails to cause a high magnitude of suction of fluid (e.g., saline, blood, clot material) through the aspiration catheter 1402 in the proximal direction. The flow sensor 1420 thus measures a flow rate that indicates that a clog is present, which can cause the thrombectomy system 1400 to enter a passive state (as indicated in FIG. 14B by decision block 1440 and the “Yes” arrow extending therefrom toward action block 1442).

### Example Embodiments

Embodiments disclosed herein can include those in the following numbered clauses:

Clause 1. A thrombectomy system, comprising: an aspiration catheter configured for advancement through vasculature of a subject to facilitate clot removal from the vasculature of the subject; a fluid jet proximate to a distal end of the aspiration catheter; and a flow direction sensor positioned on the aspiration catheter proximate to the fluid jet, wherein the flow direction sensor is configured to detect a flow direction of fluid at a distal region of the aspiration catheter during operation of a clot removal state of the thrombectomy system, wherein the flow direction of the fluid at the distal region of the aspiration catheter indicates a clog state of the aspiration catheter.

Clause 2. The thrombectomy system of clause 1, wherein the flow direction sensor comprises an ultrasonic flow meter that includes a plurality of ultrasonic transducers.

Clause 3. The thrombectomy system of clause 1, wherein the flow direction sensor comprises an optical flow sensor that includes a light emitter and a light detector.

Clause 4. The thrombectomy system of clause 3, wherein the light emitter and the light detector are positioned angularly offset about a wall of the aspiration catheter.

Clause 5. The thrombectomy system of clause 1, wherein the flow direction sensor comprises a temperature sensor.

Clause 6. The thrombectomy system of clause 5, wherein the temperature sensor comprises a micro-electromechanical system (MEMS) thermocouple.

Clause 7. The thrombectomy system of clause 1, wherein the flow direction sensor comprises an electromagnetic flow meter that includes a plurality of coils and a plurality of electrodes.

Clause 8. The thrombectomy system of clause 1, wherein the thrombectomy system is configured to present an alert on a user interface associated with the thrombectomy system after determining that the flow direction of the fluid at the distal region of the aspiration catheter during operation of the clot removal state satisfies one or more conditions.

Clause 9. The thrombectomy system of clause 8, wherein the alert comprises a directive to deactivate the clot removal state of the thrombectomy system.

Clause 10. The thrombectomy system of clause 8, wherein the alert comprises an indication of clogging of the aspiration catheter.

Clause 11. The thrombectomy system of clause 8, wherein the one or more conditions comprise the flow direction of the fluid at the distal region of the aspiration catheter during operation of the clot removal state indicating fluid flow in a distal direction.

Clause 12. The thrombectomy system of clause 1, wherein the thrombectomy system is configured to selectively deactivate the clot removal state after determining that the flow direction of the fluid at the distal region of the aspiration catheter during operation of the clot removal state satisfies one or more conditions.

Clause 13. The thrombectomy system of clause 12, wherein the one or more conditions comprise the flow direction of the fluid at the distal region of the aspiration catheter during operation of the clot removal state indicating fluid flow in a distal direction.

Clause 14. A thrombectomy system, comprising: an aspiration catheter configured for advancement through vasculature of a subject to facilitate clot removal from the vasculature of the subject; a fluid jet proximate to a distal end of the aspiration catheter; and a flow sensor positioned on the aspiration catheter proximal to the fluid jet, wherein the flow sensor is configured to detect a flow rate of fluid within an aspiration lumen of the aspiration catheter during operation of a clot removal state of the thrombectomy system, wherein the flow rate of the fluid within the aspiration lumen indicates a clog state of the aspiration catheter.

Clause 15. The thrombectomy system of clause 14, wherein the flow sensor comprises an ultrasonic flow meter that includes a plurality of ultrasonic transducers.

Clause 16. The thrombectomy system of clause 14, wherein the flow sensor comprises an electromagnetic flow meter that includes a plurality of coils and a plurality of electrodes.

Clause 17. The thrombectomy system of clause 14, wherein the flow sensor comprises a vortex shedding flow meter that includes a bluff body and one or more pressure sensors.

Clause 18. The thrombectomy system of clause 14, wherein the flow sensor comprises a thermal flow meter that includes a plurality of temperature sensors.

Clause 19. The thrombectomy system of clause 14, wherein the flow sensor comprises a differential pressure flow meter that includes a restrictive element.

Clause 20. The thrombectomy system of clause 14, wherein the thrombectomy system is configured to present an alert on a user interface associated with the thrombectomy system after determining that the flow rate of the fluid within the aspiration lumen of the aspiration catheter during operation of the clot removal state satisfies one or more conditions.

Clause 21. The thrombectomy system of clause 20, wherein the alert comprises a directive to deactivate the clot removal state of the thrombectomy system.

Clause 22. The thrombectomy system of clause 20, wherein the alert comprises an indication of clogging of the aspiration catheter.

Clause 23. The thrombectomy system of clause 20, wherein the one or more conditions comprise the flow rate of the fluid within the aspiration lumen of the aspiration catheter during operation of the clot removal state being above a threshold flow rate.

Clause 24. The thrombectomy system of clause 14, wherein the thrombectomy system is configured to selectively deactivate the clot removal state after determining that the flow rate of the fluid within the aspiration lumen of the aspiration catheter during operation of the clot removal state satisfies one or more conditions.

Clause 25. The thrombectomy system of clause 24, wherein the one or more conditions comprise the flow rate of the fluid within the aspiration lumen of the aspiration catheter during operation of the clot removal state being above a threshold flow rate.

### Additional Details Related to Implementing the Disclosed Embodiments

The principles disclosed herein may be implemented in various formats. For example, at least some techniques discussed herein may be performed as a method that includes various acts for achieving particular results or benefits. In some instances, the techniques discussed herein are represented in computer-executable instructions that may be stored on one or more hardware storage devices. The computer-executable instructions may be executable by one or more processors to carry out (or to configure a system to carry out) the disclosed techniques. In some embodiments, a system may be configured to send the computer-executable instructions to a remote device to configure the remote device for carrying out the disclosed techniques.

Systems for implementing the disclosed embodiments may include various components, such as, by way of non-limiting example, processor(s), storage, sensor(s), I/O system(s), communication system(s), etc.

The processor(s) may comprise one or more sets of electronic circuitries that include any number of logic units, registers, and/or control units to facilitate the execution of computer-readable instructions (e.g., instructions that form a computer program). Such computer-readable instructions may be stored within storage. The storage may comprise physical system memory and may be volatile, non-volatile, or some combination thereof. Furthermore, storage may comprise local storage, remote storage (e.g., accessible via communication system(s) or otherwise), or some combination thereof.

In some implementations, the processor(s) may comprise or be configurable to execute any combination of software and/or hardware components that are operable to facilitate processing using machine learning models or other artificial intelligence-based structures/architectures. Artificial intelligence-based structures/architectures may take on any suitable form, such as by comprising or utilizing hardware components and/or computer-executable instructions operable to carry out function blocks and/or processing layers configured in the form of, by way of non-limiting example, single-layer neural networks, feed forward neural networks, radial basis function networks, deep feed-forward networks, recurrent neural networks, long-short term memory (LSTM) networks, gated recurrent units, autoencoder neural networks, variational autoencoders, denoising autoencoders, sparse autoencoders, Markov chains, Hopfield neural networks, Boltzmann machine networks, restricted Boltzmann machine networks, deep belief networks, deep convolutional networks (or convolutional neural networks), deconvolutional neural networks, deep convolutional inverse graphics networks, generative adversarial networks, liquid state machines, extreme learning machines, echo state networks, deep residual networks, Kohonen networks, support vector machines, neural Turing machines, and/or others.

In some instances, actions performable by a system may rely at least in part on communication system(s) for receiving information from remote system(s), which may include, for example, separate systems or computing devices, sensors, and/or others. The communications system(s) may comprise any combination of software or hardware components that are operable to facilitate communication between on-system components/devices and/or with off-system components/devices. For example, the communications system(s) may comprise ports, buses, or other physical connection apparatuses for communicating with other devices/components. Additionally, or alternatively, the communications system(s) may comprise systems/components operable to communicate wirelessly with external systems and/or devices through any suitable communication channel(s), such as, by way of non-limiting example, Bluetooth, ultra-wideband, WLAN, infrared communication, and/or others.

A system may comprise or be in communication with sensor(s). Sensor(s) may comprise any device for capturing or measuring data representative of perceivable phenomenon. By way of non-limiting example, the sensor(s) may comprise one or more image sensors, microphones, thermometers, barometers, magnetometers, accelerometers, gyroscopes, and/or others.

Furthermore, a system may comprise or be in communication with I/O system(s). I/O system(s) may include any type of input or output device such as, by way of non-limiting example, a touch screen, a mouse, a keyboard, a controller, and/or others, without limitation. For example, the 1/O system(s) may include a display system that may comprise any number of display panels, optics, laser scanning display assemblies, and/or other components. One will appreciate, in view of the present disclosure, that the sensor(s) may, in some instances, be utilized as I/O system(s).

Disclosed embodiments may comprise or utilize a special purpose or general-purpose computer including computer hardware, as discussed in greater detail below. Disclosed embodiments also include physical and other computer-readable media for carrying or storing computer-executable instructions and/or data structures. Such computer-readable media can be any available media that can be accessed by a general-purpose or special-purpose computer system. Computer-readable media that store computer-executable instructions in the form of data are one or more “computer-readable recording media,” “physical computer storage media,” or “hardware storage device(s).” Computer-readable media that merely carry computer-executable instructions without storing the computer-executable instructions are “transmission media.” Thus, by way of example and not limitation, the current embodiments can comprise at least two distinctly different kinds of computer-readable media: computer storage media and transmission media.

Computer storage media (aka “hardware storage device”) are computer-readable hardware storage devices, such as RAM, ROM, EEPROM, CD-ROM, solid state drives (“SSD”) that are based on RAM, Flash memory, phase-change memory (“PCM”), or other types of memory, or other optical disk storage, magnetic disk storage or other magnetic storage devices, or any other medium that can be used to store desired program code means in hardware in the form of computer-executable instructions, data, or data structures and that can be accessed by a general-purpose or special-purpose computer.

A “network” may comprise one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmission media can include a network and/or data links which can be used to carry program code in the form of computer-executable instructions or data structures and which can be accessed by a general purpose or special purpose computer. Combinations of the above are also included within the scope of computer-readable media.

Further, upon reaching various computer system components, program code means in the form of computer-executable instructions or data structures can be transferred automatically from transmission computer-readable media to physical computer-readable storage media (or vice versa). For example, computer-executable instructions or data structures received over a network or data link can be buffered in RAM within a network interface module (e.g., a “NIC”), and then eventually transferred to computer system RAM and/or to less volatile computer-readable physical storage media at a computer system. Thus, computer-readable physical storage media can be included in computer system components that also (or even primarily) utilize transmission media.

Computer-executable instructions comprise, for example, instructions and data which cause a general-purpose computer, special purpose computer, or special purpose processing device to perform a certain function or group of functions. The computer-executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Disclosed embodiments may comprise or utilize cloud computing. A cloud model can be composed of various characteristics (e.g., on-demand self-service, broad network access, resource pooling, rapid elasticity, measured service, etc.), service models (e.g., Software as a Service (“SaaS”), Platform as a Service (“PaaS”), Infrastructure as a Service (“IaaS”), and deployment models (e.g., private cloud, community cloud, public cloud, hybrid cloud, etc.).

Those skilled in the art will appreciate that the invention may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, pagers, routers, switches, wearable devices, and the like. The invention may also be practiced in distributed system environments where multiple computer systems (e.g., local and remote systems), which are linked through a network (either by hardwired data links, wireless data links, or by a combination of hardwired and wireless data links), perform tasks. In a distributed system environment, program modules may be located in local and/or remote memory storage devices.

Alternatively, or in addition, the functionality described herein can be performed, at least in part, by one or more hardware logic components. For example, and without limitation, illustrative types of hardware logic components that can be used include Field-programmable Gate Arrays (FPGAs), Program-specific Integrated Circuits (ASICs), Application-specific Standard Products (ASSPs), System-on-a-chip systems (SOCs), Complex Programmable Logic Devices (CPLDs), central processing units (CPUs), graphics processing units (GPUs), and/or others.

As used herein, the terms “executable module,” “executable component,” “component,” “module,” or “engine” can refer to hardware processing units or to software objects, routines, or methods that may be executed on one or more computer systems. The different components, modules, engines, and services described herein may be implemented as objects or processors that execute on one or more computer systems (e.g., as separate threads).

It is contemplated that various combinations or sub-combinations of the specific features and aspects of the embodiments disclosed above may be made and still fall within one or more of the embodiments. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed embodiments. Thus, it is intended that the scope of the present disclosure herein disclosed should not be limited by the particular disclosed embodiments described above. Moreover, while the present disclosure is susceptible to various modifications, and alternative forms, specific examples thereof have been shown in the drawings and are herein described in detail. It should be understood, however, that the present disclosure is not to be limited to the particular forms or methods disclosed, but to the contrary, the present disclosure is to cover all modifications, equivalents, and alternatives falling within the spirit and scope of the various embodiments described and the appended claims.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “approximately”, “about”, and “substantially” as used herein include the recited numbers (e.g., about 10%=10%), and also represent an amount close to the stated amount that still performs a desired function or achieves a desired result. For example, the terms “approximately”, “about”, and “substantially” may refer to an amount that is within less than 10% of, within less than 5% of, within less than 1% of, within less than 0.1% of, and within less than 0.01% of the stated amount.

For purposes of the present disclosure and appended claims, the conjunction “or” is to be construed inclusively (e.g., “an apple or an orange” would be interpreted as “an apple, or an orange, or both”; e.g., “an apple, an orange, or an avocado” would be interpreted as “an apple, or an orange, or an avocado, or any two, or all three”), unless: (i) it is explicitly stated otherwise, e.g., by use of “either . . . or,” “only one of,” or similar language; or (ii) two or more of the listed alternatives are mutually exclusive within the particular context, in which case “or” would encompass only those combinations involving non-mutually-exclusive alternatives. For purposes of the present disclosure and appended claims, the words “comprising,” “including,” “having,” and variants thereof, wherever they appear, shall be construed as open-ended terminology, with the same meaning as if the phrase “at least” were appended after each instance thereof.

We claim:

1. A thrombectomy system, comprising:
an aspiration catheter configured for advancement through vasculature of a subject to facilitate clot removal from the vasculature of the subject;
a fluid jet proximate to a distal end of the aspiration catheter; and
a flow direction sensor positioned on the aspiration catheter proximate to the fluid jet, wherein the flow direction sensor is configured to detect a flow direction of fluid at a distal region of the aspiration catheter during operation of a clot removal state of the thrombectomy system, wherein the flow direction of the fluid at the distal region of the aspiration catheter indicates a clog state of the aspiration catheter.

2. The thrombectomy system of claim 1, wherein the flow direction sensor comprises an ultrasonic flow meter that includes a plurality of ultrasonic transducers.

3. The thrombectomy system of claim 1, wherein the flow direction sensor comprises an optical flow sensor that includes a light emitter and a light detector.

4. The thrombectomy system of claim 3, wherein the light emitter and the light detector are positioned angularly offset about a wall of the aspiration catheter.

5. The thrombectomy system of claim 1, wherein the flow direction sensor comprises a temperature sensor.

6. The thrombectomy system of claim 5, wherein the temperature sensor comprises a micro-electromechanical system (MEMS) thermocouple.

7. The thrombectomy system of claim 1, wherein the flow direction sensor comprises an electromagnetic flow meter that includes a plurality of coils and a plurality of electrodes.

8. The thrombectomy system of claim 1, wherein the thrombectomy system is configured to present an alert on a user interface associated with the thrombectomy system after determining that the flow direction of the fluid at the distal region of the aspiration catheter during operation of the clot removal state satisfies one or more conditions.

9. The thrombectomy system of claim 8, wherein the alert comprises a directive to deactivate the clot removal state of the thrombectomy system.

10. The thrombectomy system of claim 8, wherein the one or more conditions comprise the flow direction of the fluid at the distal region of the aspiration catheter during operation of the clot removal state indicating fluid flow in a distal direction.

11. The thrombectomy system of claim 1, wherein the thrombectomy system is configured to selectively deactivate the clot removal state after determining that the flow direction of the fluid at the distal region of the aspiration catheter during operation of the clot removal state satisfies one or more conditions.

12. A thrombectomy system, comprising:
an aspiration catheter configured for advancement through vasculature of a subject to facilitate clot removal from the vasculature of the subject;
a fluid jet proximate to a distal end of the aspiration catheter; and
a flow sensor positioned on the aspiration catheter proximal to the fluid jet, wherein the flow sensor is configured to detect a flow rate of fluid within an aspiration lumen of the aspiration catheter during operation of a clot removal state of the thrombectomy system, wherein the flow rate of the fluid within the aspiration lumen indicates a clog state of the aspiration catheter.

13. The thrombectomy system of claim 12, wherein the flow sensor comprises an ultrasonic flow meter that includes a plurality of ultrasonic transducers.

14. The thrombectomy system of claim 12, wherein the flow sensor comprises an electromagnetic flow meter that includes a plurality of coils and a plurality of electrodes.

15. The thrombectomy system of claim 12, wherein the flow sensor comprises a vortex shedding flow meter that includes a bluff body and one or more pressure sensors.

16. The thrombectomy system of claim 12, wherein the flow sensor comprises a thermal flow meter that includes a plurality of temperature sensors.

17. The thrombectomy system of claim 12, wherein the flow sensor comprises a differential pressure flow meter that includes a restrictive element.

18. The thrombectomy system of claim 12, wherein the thrombectomy system is configured to present an alert on a user interface associated with the thrombectomy system after determining that the flow rate of the fluid within the aspiration lumen of the aspiration catheter during operation of the clot removal state satisfies one or more conditions.

19. The thrombectomy system of claim 18, wherein the one or more conditions comprise the flow rate of the fluid within the aspiration lumen of the aspiration catheter during operation of the clot removal state being above a threshold flow rate.

20. The thrombectomy system of claim 12, wherein the thrombectomy system is configured to selectively deactivate the clot removal state after determining that the flow rate of the fluid within the aspiration lumen of the aspiration catheter during operation of the clot removal state satisfies one or more conditions.
